(19) United States
(12) Patent Application Publication
(10) Pub. No.: US 2008/0025952 A1
(43) Pub. Date: Jan. 31, 2008

(54) Methods for targeted deliver of genetic material to the liver

(76) Inventors: Ronald K. Scheule, Hopkinton, MA (US); Bradley L. Hodges, Milford, MA (US)

Correspondence Address: GENZYME CORPORATION;LEGAL DEPARTMENT, 15 PLEASANT ST CONNECTOR, FRAMINGHAM, MA, 01701-9322, US

(21) Appl. No.: 11/804,291

(22) Filed: May 17, 2007

Related U.S. Application Data
(63) Continuation of application No. PCT/US05/43590, filed on Dec. 1, 2005.
(60) Provisional application No. 60/632,359, filed on Dec. 1, 2004.

(51) Int. Cl. A61K 48/00; A61K 31/7088; A61P 3/00

(52) U.S. Cl. 424/93.2; 514/44

(57) ABSTRACT

The present invention provides methods for enhanced delivery of various therapeutic agents, such as gene therapy agents, to the vasculature of a target organ in a mammalian subject. The methods for targeted gene therapy in the mammalian liver as a whole, or in a single hepatic lobe, are disclosed. The disclosed methods rely on minimally invasive catheter-based procedures wherein a target organ is isolated and treated locally with a gene therapy agent. The methods offer more efficient and localized transfection of tissue and are well-suited for gene therapy in human subjects.

### FIELD OF THE INVENTION

The present invention relates to methods for balloon catheter delivery of genetic material to a target organ of a living subject.

### BACKGROUND OF THE INVENTION

Gene therapy is the intracellular delivery of exogenous genetic material that corrects an existing defect or provides a new beneficial function to the cells. The liver is an important target organ for gene therapy because of its central role in metabolism and production of serum proteins. There are a large number of known diseases, some of which are caused by defects in liver-specific gene products that could benefit from liver production of a secreted protein. Familial hypercholesterolemia, hemophilia, Gaucher's and Fabry's diseases are just a few examples. Many such diseases may be amenable to gene therapy (Siatskas et al., J. Inherit Metab. Dis. 2001, 24 (Suppl. 2): 25-41; Barranger et al., Expert Opin. Biol. Ther. 2001, 1(5): 857-867; Barranger et al., Neurochem Res. 1999, 24(5): 601-615).

Various methods have been developed to deliver exogenous genetic material to the liver using viral and non-viral vectors. Generally, each method possesses certain drawbacks. Previous attempts at delivery of genetic material using viral vectors have been complicated by neutralizing host immune responses, toxicity due to pre-existing host immunity, the need for large volumes of therapeutic agent to be injected into the subject's circulation, elevated pressures within the target organ during therapy, and difficulty targeting specific cell types within the body.

Portal injection of viral vectors has been attempted as a means of targeting of the liver. However, portal injection presents several problems. When adenoviral gene transfer vectors are injected into the portal vein of a rat, high levels of transgene expression are observed in the liver (Rosefeld et al., Science 1991, 252: 431-434), but such expression is transient and requires repeated injections. Additionally, when injected in the circulatory system of seropositive animals, viral vectors may be quickly neutralized by pre-existing antibodies. Studies of systemic injections of recombinant adenoviral vectors have shown that a neutralizing host immune response limits the effectiveness of such vectors in repeated injections (Yang et al., Proc. Natl. Acad. Sci. U.S.A. 1994, 91: 4407-4411; Kozarsky et al., J. Biol. Chem. 1994, 269:13695-13702).

In other cases, systemic or portal injection of viral vectors has been associated with dose-dependent toxicities. These toxicities are due to both the relatively large volumes of virus which must be injected and to pre-existing immunity as a result of prior environmental exposure to common viral serotypes. Therefore, it is desirable to limit both the amount of virus delivered to the subject and the degree to which the virus is exposed to the systemic circulation and hence the immune system.

Another challenge with systemic delivery of viral gene therapeutics is targeting of the therapeutics to appropriate cells within the target organ. For example, in the liver, both hepatocytes and non-hepatocytes (including Kupffer cells and other antigen-presenting cells) may be transfected. Hepatocytes are excellent protein producing cells, can secrete expressed proteins into the serum, and are often the site of loss-of-function defects. Therefore, it is desirable to maximize transfection of hepatocytes versus liver non-hepatocytes. However, with systemically administered viral gene therapy, a significant fraction of the transfected liver cells are non-hepatocytes.

There may be a negative consequence of transgene expression in non-hepatocytes, such as in antigen-presenting cells (including Kupffer cell, liver sinusoidal endothelial cell). Such expression may generate immune responses against the transgene product.

Previous attempts have been made at delivering genetic materials to isolated regions of the body using balloon occlusion catheters (U.S. Pat. No. 5,698,531). These methods are aimed at transfection of endothelial cells lining the surface of the vessel. The present invention provides a method for delivering genetic material to the parenchymal cells of an organ.

Another method for delivering genetic materials to target organs with balloon catheters has been described (WO 2004/001049). However, this method requires the use of elevated pressures within the target organ. In order to elevate pressures sufficiently, larger volumes of therapeutic agent must be injected, and these larger volumes may be disadvantageous for the reasons noted above. Furthermore, the elevated pressure may risk damaging the target organ.

### SUMMARY OF THE INVENTION

It is accordingly an object of the present invention to provide a method for delivering a viral gene therapy agent to a target organ through the venous vasculature, which drains said target organ.

It is another object of the present invention to provide a method for delivering a viral gene therapy agent to a target organ through the venous vasculature, which drains said target organ, without significantly increasing the pressure in said venous vasculature of said target organ.

It is another object of the present invention to provide a method for delivering a viral gene therapy agent to a target organ through the venous vasculature, which drains said target organ, wherein said organ is the liver and the venous vasculature is a hepatic vein, a tributary of a hepatic vein, or the inferior vena cava.

It is another object of the present invention to provide a method for delivering a viral gene therapy agent to a target organ in order to express a protein encoded by said viral gene therapy agent.

It is another object of the present invention to provide a method for delivering a viral gene therapy agent to the liver in order to express a protein encoded by said viral gene therapy agent in both hepatocytes and non-hepatocytes, wherein the fraction of hepatocytes which express the protein encoded by said viral gene among the total of hepatocytes plus non-hepatocytes expressing the protein encoded by said viral gene, is at least 0.2, at least 0.3, at least 0.4, at least 0.5, at least 0.6, at least 0.7, at least 0.8, or at least 0.9.

It is another object of the present invention to provide a method for delivering a viral gene therapy agent to a target organ in order to express a protein encoded by said viral gene therapy agent wherein the viral gene therapy agent is delivered to a subject with pre-existing immunity to said viral gene therapy agent.

In accordance with the invention, a method is provided for targeted delivery of genetic material using a balloon catheter. In one method, a balloon occlusion catheter is engaged proximally in a single hepatic vein and a therapeutic solution is delivered beyond the inflated (occluding) balloon via a catheter to the liver parenchyma through the vessels of the thus-isolated target lobe. The volume of therapeutic agent delivered is sufficiently large to perfuse the venous vasculature of the target organ, but small enough to prevent a significant rise in the venous vascular pressure or distribute the agent systemically via collateral circulation. By repositioning the balloon to occlude different vessels, multiple lobes can be treated sequentially during the same procedure. Since treatment is highly localized, various parts of a single organ can be treated in the same procedure with different therapeutic agents that may otherwise be incompatible.

In another method, venous outflow from the entire organ is temporarily occluded by the placement of balloon catheters in the inferior vena cava both proximal and distal to the hepatic venous outflow, and the gene therapy agent is injected via an endovascular catheter in the space between the inflated (occluding) balloons. Again, the volume of the viral therapeutic agent is sufficiently large as to perfuse the vasculature of the target organ, but small enough to prevent a significant rise in the vascular pressure or distribute the agent systemically via collateral circulation. When this method is used to deliver a viral gene therapy agent to the isolated liver, very effective gene transfer is achieved.

In another embodiment, the methods of the present invention may also include a “flushing” step prior to viral administration. Flushing utilizes a physiologically appropriate solution, such as saline, to perfuse or partially perfuse the isolated organ or section of the organ prior to administration of virus to reduce or eliminate pre-existing antibodies against the viral vector that might otherwise reduce the ability of the gene transfer vector to transduce or infect the target cells.

In another embodiment, when the liver is the target organ, the flushing step prior to viral administration may increase the number and proportion of hepatocytes transfected. In another preferred embodiment, the flushing step prior to viral administration increases the number and proportion of hepatocytes transfected in an animal with pre-existing immunity to the viral vector administered.

In yet another embodiment, the methods of the present invention may include an extended residence, or dwell time, for the viral gene therapy agent in the target organ. The extended dwell time may increase the number and proportion of hepatocytes transfected in an animal without increasing the acute toxicity associated with the instant methods.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

The accompanying drawings, which are incorporated in and constitute a part of this specification, illustrate several embodiments of the invention and together with the description, serve to explain the principles of the invention.

### BRIEF DESCRIPTION OF THE DRAWINGS

FIG. 1 illustrates a balloon catheter (4) occluding a hepatic vein (3). The catheter is inserted through the jugular vein (5), passed through the superior vena cava (1), through the heart (2), and into a hepatic vein (3).

FIG. 2 illustrates a fluoroscopic spot image of balloon catheter administration of virus through a hepatic vein in the rabbit model. Dotted lines indicate the vena cava and four major hepatic veins. The catheter can be seen descending through the superior vena cava into the right caudal lobe, where the occlusion balloon, inflated with contrast agent, blocks outflow from the vein. The injected solution, which contains contrast agent, can be seen highlighting the venous branching of this lobe (dotted circle). The image was captured just prior to virus injection and illustrates the patency of the balloon-mediated occlusion of the selected hepatic vein.

FIG. 3 illustrates a fluoroscopic image of a balloon catheter (4) within a hepatic vein (HV Block.) A second balloon (6) is inflated within the inferior vena cava (IVC Block.)

FIG. 4 illustrates a dual-balloon technique for isolating the venous drainage of the liver. One balloon (7) is advanced towards the liver via the superior vena cava (1) after insertion through the jugular vein (5). A second occluding balloon (8) is inserted through the inferior vena cava (18). The first balloon (7) occludes a portion of the inferior vena cava which is above the hepatic veins (3, 10, 11), and the second balloon (8) occludes a portion of the inferior vena cava (18) which is below the hepatic veins (3, 10, 11). A third catheter (9) is used to infuse a therapeutic agent.

FIG. 5 illustrates another dual-balloon technique. The balloons (12, 13) are used for occlusion, and a lumen may extend through one or both of the balloons (12, 13) so that the therapeutic agent can be injected through a hole in the one or both of the catheter tips (14, 15).

FIG. 6 illustrates the distribution of β-galactosidase expression after administering 1.5×10¹² vp/kg of Ad2-βgal as a function of a 3 ml (A, B, C), 8 ml (D, E, F), or 20 ml (G, H, I) injection volume using a balloon catheter and rabbits naive to Ad2. Expression as evaluated by immunohistochemistry is shown at 10× for both the injected (Lobe 1) and an un-injected (Lobe 4) lobe (A, B, D, E, G, H) with photomicrographs typical of 2 injected animals. Expression of bacterial β-galactosidase in 3 cores per lobe is shown schematically (C, F, & I); the injected lobe is shaded. The numbers represent expression in relative light units of β-galactosidase/mg protein. For simplicity, lobes are numbered. Note that the injected lobe (Lobe 1) is the circumscribed lobe shown in FIG. 2.

FIG. 7 illustrates the distribution of β-galactosidase expression after systemically administering 1.5×10¹² vp/kg of Ad2-βgal in a volume of 8 ml to rabbits naive to Ad2 (A, C, E) and to rabbits passively immunized with human serum containing anti-Ad2 antibodies (B, D, F). Immunohistochemical localization of β-galactosidase expression in Lobe 1 is shown at 10× and 40× in photomicrographs typical of 2-3 injected animals. Schematic of the distribution of β-galactosidase expression determined by ELISA is shown for naive (E) and passively immunized (F) rabbits (since this is a systemic administration, there is no distinction between lobes.)

FIG. 8 illustrates the distribution of β-galactosidase expression after administering 1.5×10¹² vp/kg of Ad2-βgal to naive rabbits (A-E) and to rabbits passively immunized to Ad2 (F-J) using a balloon catheter and a volume of 8 ml. Expression as evaluated by immunohistochemistry is shown at 10× and 40× for both the injected (Lobe 1; A, C, F, H) and an un-injected (Lobe 4; B, D, G, I) lobe in photomicrographs typical of 3 injected rabbits. Expression as determined by ELISA in each lobe is shown schematically; the injected lobe is shaded. The numbers represent the average expression in units of pg β-galactosidase/μg protein from 3 tissue cores per lobe.

FIG. 9 quantifies the number of hepatocytes and non-hepatocytes expressing the transgene after delivery of a viral gene therapy agent according to the method of the present invention. Metamorph quantitation of the number of β-galactosidase positive hepatocytes (filled bars) and non-hepatocytes (open bars) per 1 mm² field of each liver lobe following local (A, B, and C) or systemic (D and E) delivery of an identical amount of virus (1.5×10¹² vp/kg of Ad2-βgal) in an identical volume (8 ml) into naive rabbits (A and D) or rabbits passively immunized with human serum containing anti-Ad2 antibodies (B, C and E). The injected lobe of animals in (C) was flushed with 20 ml of saline immediately prior to administration of 8 ml of virus. Numbers in parentheses represent the fraction of β-gal positive hepatocytes among all β-gal positive cells within each given lobe. Lobe numbers 1 and 4 correspond to those shown schematically in FIGS. 6-8. (A, B, C & E, N=30 fields, D; N=45 fields).

FIG. 10 illustrates human α-galactosidase A expression over 84 days after administering 5×10¹² drp of AAV2DC190HAGAL virus in a total volume of 8 ml via local delivery to the liver using balloon catheter-mediated delivery to three naive rabbits. Expression was detected in two out of the three rabbits for the entire 84 day period and in the third rabbit starting between day 7 and 14 for the remainder of the 84 day period.

FIG. 11A illustrates the transfected hepatocyte fraction, which is the proportion of hepatocytes expressing the transgene as compared to the total cells expressing the transgene, after local catheter-based delivery of the Ad2βgal virus (1.5×10¹² vp/kg) in an 8 ml volume into naive rabbits with a 4 minute dwell time according to the method of the present invention. Metamorph quantitation of the number of β-galactosidase positive hepatocytes and non-hepatocytes was performed on three sections of both the injected liver lobe and an un-injected liver lobe. Each bar represents the hepatocyte fraction [transfected hepatocytes/(transfected hepatocytes+transfected non-hepatocyte cells)] analysis of one liver section. Bars for 8-20 and 8-22 represent rabbits treated with the 4 minute dwell time while bars for 4, 5, and 1 represent rabbits treated using a 1 minute dwell time from a prior experiment using identical protocols except for the dwell time.

FIG. 11B illustrates the distribution of β-galactosidase expression as determined by ELISA after administering 1.5×10¹² vp/kg of Ad2-βgal to rabbits using a balloon catheter and a volume of 8 ml with 1) a 4 minute dwell time (8-20 and 8-22) and from a prior experiment of otherwise identical conditions with 2) a 1 minute dwell time (4, 5, and 1). Expression was measured in the four liver lobes, the lung, kidney, and spleen. Three tissue cores were taken from each liver lobe with which to measure expression; they were proximal, medial and distal in the lobe to the point of entry of a lobar hepatic vein into the vena cava. RCP, RCM, RCD refers to the right lateral lobe (injected lobe), proximal, medial and distal, respectively. RLP, RLM, RLD refer to right medial lobe, proximal, medial and distal, respectively. MP, MM, MD refer to left medial lobe, proximal, medial and distal, respectively. LP, LM, LD refer to left lateral lobe, proximal, medial and distal, respectively.

### DESCRIPTION OF THE EMBODIMENTS

Reference will now be made in detail to the present embodiments of the invention, examples of which are illustrated in the accompanying drawings. Wherever possible, the same reference numbers will be used throughout the drawings to refer to the same or like parts.

The practice of the present invention employs, unless otherwise indicated, conventional techniques of molecular biology (including recombinant DNA techniques), microbiology, cell biology, biochemistry and immunology, which are within the skill of the art. Such techniques are explained fully in the literature, such as, Molecular Cloning: A Laboratory Manual, Second Edition (Sambrook et al., 1989); Current Protocols In Molecular Biology (F. M. Ausubel et al., eds., 1987); Oligonucleotide Synthesis (M. J. Gait, ed., 1984); Animal Cell Culture (R. I. Freshney, ed., 1987); Methods In Enzymology (Academic Press, Inc.); Handbook Of Experimental Immunology (D. M. Wei & C. C. Blackwell, eds.); Gene Transfer Vectors For Mammalian Cells (J. M. Miller & M. P. Calos, eds., 1987); PCR: The Polymerase Chain Reaction, (Mullis et al., eds., 1994); Current Protocols In Immunology (J. E. Coligan et al., eds., 1991); Antibodies: A Laboratory Manual (E. Harlow and D. Lane eds. (1988)); and PCR 2: A Practical Approach (M. J. MacPherson, B. D. Hames and G. R. Taylor eds. (1995)).

The term “transgene” refers to a polynucleotide that is introduced into the cells of a tissue or an organ and is capable of being expressed under appropriate conditions, or otherwise conferring a beneficial property to the cells. A transgene is selected based upon a desired therapeutic outcome. It may encode, for example, hormones, enzymes, receptors, or other proteins of interest. It may also encode a small interfering RNA (siRNA) or antisense RNA for the purpose of decreasing or eliminating expression of an endogenous or exogenously-administered gene. For instance, in the treatment of familial hypercholesterolemia, one may use a transgene encoding LDL receptor (Kobayashi et al., J. Biol. Chem. 271: 6852-6860).

The term “transfection” is used interchangeably with the term “gene transfer” and “transduction” and means the intracellular introduction of a transgene. “Transfection efficiency” refers to the relative amount of the transgene taken up by the cells subjected to transfection. In practice, transfection efficiency is estimated by the amount of the reporter gene product expressed following the transfection procedure.

Gene delivery, gene transfer, and the like as used herein, are terms referring to the introduction of an exogenous polynucleotide (sometimes referred to as a “transgene”) into a host cell. The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon or a nuclear or mitochondrial chromosome. A number of viral vectors are known to be capable of mediating transfer of genes to mammalian cells, as is known in the art and described herein.

The exogenous polynucleotide is inserted into a viral vector for delivery to the host via the method of the instant invention. Many viral vectors are known, including many species of each, and many have been studied for gene therapy purposes. The most commonly used viral vectors include those derived from adenoviruses, adeno-associated viruses [AAV] and retroviruses, including lentiviruses, such as human immunodeficiency virus [HIV].

The term “virus” refers to an agent capable of transferring DNA or RNA to a cell and which is an obligate intracellular organism of living but non-cellular nature, consisting of DNA or RNA and a protein coat. Virus does not include naked DNA, naked RNA, plasmid DNA without a protein coat, or RNA without a protein coat. Examples of viruses which may be applicable to the methods of the present invention include adenoviruses, adeno-associated virus, alphaviruses, baculoviruses, hepadenaviruses, baculoviruses, poxviruses, herpesviruss, retroviruses, lentiviruses, orthomyxoviruses, papovaviruses, paramyxoviruses, and parvoviruses. In addition, hybrid viruses produced from combinations of any of these viruses may be used. These include adenovirus vectors, adeno-associated virus vectors, retrovirus vectors, lentivirus vectors, and plasmid vectors. Exemplary types of viruses include HSV (herpes simplex virus), AAV (adeno associated virus), HIV (human immunodeficiency virus), BIV (bovine immunodeficiency virus), and MLV (murine leukemia virus).

In selecting a virus for delivery to a particular mammal using the methods of the instant invention, a specific serotype of a particular virus may be selected with the mammal to be treated in mind. The serotype may be selected from one that was isolated in such a mammal and/or which may have an enhanced tropism for the particular target organ of a particular target mammal to be treated.

Alternatively, in selecting a virus for delivery to a particular mammal using the methods of the instant invention, a serotype that was not isolated in the particular species of the mammal may be selected.

Adenovirus is a non-enveloped, nuclear DNA virus with a genome of about 36 kb, which has been well-characterized through studies in classical genetics and molecular biology (Hurwitz, M. S., Adenoviruses Virology, 3rd edition, Fields et al., eds., Raven Press, New York, 1996; Hitt, M. M. et al., Adenovirus Vectors, The Development of Human Gene Therapy, Friedman, T. ed., Cold Spring Harbor Laboratory Press, New York 1999). The viral genes are classified into early (designated E1-E4) and late (designated L1-L5) transcriptional units, referring to the generation of two temporal classes of viral proteins. The demarcation of these events is viral DNA replication. The human adenoviruses are divided into numerous serotypes (approximately 47, numbered accordingly and classified into 6 groups: A, B, C, D, E and F), based upon properties including hemagglutination of red blood cells, oncogenicity, DNA and protein amino acid compositions and homologies, and antigenic relationships.

Recombinant adenoviral vectors have several advantages for use as gene delivery vehicles, including tropism for both dividing and non-dividing cells, minimal pathogenic potential, ability to replicate to high titer for preparation of vector stocks, and the potential to carry large inserts (Berkner, K. L., Curr. Top. Micro. Immunol. 158:39-66, 1992; Jolly, D., Cancer Gene Therapy 1:51-64 1994). Adenoviral vectors with deletions of various adenoviral gene sequences, such as pseudoadenoviral vectors (PAVs) and partially-deleted adenoviral (termed “DeAd”), have been designed to take advantage of the desirable features of adenovirus which render it a suitable vehicle for delivery of nucleic acids to recipient cells.

In particular, pseudoadenoviral vectors (PAVs), also known as ‘gutless adenovirus’ or mini-adenoviral vectors, are adenoviral vectors derived from the genome of an adenovirus that contain minimal cis-acting nucleotide sequences required for the replication and packaging of the vector genome and which can contain one or more transgenes (See, U.S. Pat. No. 5,882,877 which covers pseudoadenoviral vectors (PAV) and methods for producing PAV, incorporated herein by reference). PAVs have been designed to take advantage of the desirable features of adenovirus which render it a suitable vehicle for gene delivery. While adenoviral vectors can generally carry inserts of up to 8 kb in size by the deletion of regions which are dispensable for viral growth, maximal carrying capacity can be achieved with the use of adenoviral vectors containing deletions of most viral coding sequences, including PAVs. See U.S. Pat. No. 5,882,877 of Gregory et al.; Kochanek et al., Proc. Natl. Acad. Sci. USA 93:5731-5736, 1996; Parks et al., Proc. Natl. Acad. Sci. USA 93:13565-13570, 1996; Lieber et al., J. Virol. 70:8944-8960, 1996; Fisher et al., Virology 217:11-22, 1996; U.S. Pat. No. 5,670,488; PCT Publication No. WO96/33280, published Oct. 24, 1996; PCT Publication No. WO96/40955, published Dec. 19, 1996; PCT Publication No. WO97/25446, published Jul. 19, 1997; PCT Publication No. WO95/29993, published Nov. 9, 1995; PCT Publication No. WO97/00326, published Jan. 3, 1997; Morral et al., Hum. Gene Ther. 10:2709-2716, 1998. Such PAVs, which can accommodate up to about 36 kb of foreign nucleic acid, are advantageous because the carrying capacity of the vector is optimized, while the potential for host immune responses to the vector or the generation of replication-competent viruses is reduced. PAV vectors contain the 5′ inverted terminal repeat (ITR) and the 3′ ITR nucleotide sequences that contain the origin of replication, and the cis-acting nucleotide sequence required for packaging of the PAV genome, and can accommodate one or more transgenes with appropriate regulatory elements, e.g. promoter, enhancers, etc.

Other, partially deleted adenoviral vectors provide a partially-deleted adenoviral (termed “DeAd”) vector in which the majority of adenoviral early genes required for virus replication are deleted from the vector and placed within a producer cell chromosome under the control of a conditional promoter. The deletable adenoviral genes that are placed in the producer cell may include E1A/E1B, E2, E4 (only ORF6 and ORF6/7 need be placed into the cell), pIX and pIVa2. E3 may also be deleted from the vector, but since it is not required for vector production, it can be omitted from the producer cell. The adenoviral late genes, normally under the control of the major late promoter (MLP), are present in the vector, but the MLP may be replaced by a conditional promoter.

Conditional promoters suitable for use in PAV or DeAd viral vectors and producer cell lines include those with the following characteristics: low basal expression in the uninduced state, such that cytotoxic or cytostatic adenovirus genes are not expressed at levels harmful to the cell; and high level expression in the induced state, such that sufficient amounts of viral proteins are produced to support vector replication and assembly. Preferred conditional promoters suitable for use in DeAd vectors and producer cell lines include the dimerizer gene control system, based on the immunosuppressive agents FK506 and rapamycin, the ecdysone gene control system and the tetracycline gene control system. Also useful in the present invention may be the GeneSwitch™ technology [Valentis, Inc., Woodlands, Tex.] described in Abruzzese et al., Hum. Gene Ther. 1999 10:1499-507, the disclosure of which is hereby incorporated herein by reference. The partially deleted adenoviral expression system is further described in WO99/57296, the disclosure of which is hereby incorporated by reference herein.

Adeno-associated virus (AAV) is a single-stranded human DNA parvovirus whose genome has a size of 4.6 kb. The AAV genome contains two major genes: the rep gene, which codes for the rep proteins (Rep 76, Rep 68, Rep 52, and Rep 40) and the cap gene, which codes for AAV replication, rescue, transcription and integration, while the cap proteins form the AAV viral particle. AAV derives its name from its dependence on an adenovirus or other helper viruses (e.g., herpesvirus) to supply essential gene products that allow AAV to undergo a productive infection, i.e., reproduce itself in the host cell. In the absence of helper virus, AAV integrates as a provirus into the host cell's chromosome, until it is rescued by superinfection of the host cell with a helper virus, usually adenovirus (Muzyczka, Curr. Top. Micro. Immunol. 158:97-127, 1992).

Interest in AAV as a gene transfer vector results from several unique features of its biology. At both ends of the AAV genome is a nucleotide sequence known as an inverted terminal repeat (ITR), which contains the cis-acting nucleotide sequences required for virus replication, rescue, packaging and integration. The integration function of the ITR mediated by the rep protein in trans permits the AAV genome to integrate into a cellular chromosome after infection, in the absence of helper virus. This unique property of the virus has relevance to the use of AAV in gene transfer, as it allows for integration of a recombinant AAV containing a gene of interest into the cellular genome. Therefore, stable genetic transformation, ideal for many of the goals of gene transfer, may be achieved by use of rAAV vectors. Furthermore, the site of integration for AAV is well-established and has been localized to chromosome 19 of humans (Kotin et al., Proc. Natl. Acad. Sci. 87:2211-2215, 1990). This predictability of integration site reduces the danger of random insertional events into the cellular genome that may activate or inactivate host genes or interrupt coding sequences, consequences that can limit the use of vectors whose integration of AAV, removal of this gene in the design of rAAV vectors may result in the altered integration patterns that have been observed with rAAV vectors (Ponnazhagan et al., Hum Gene Ther. 8:275-284, 1997).

There are other advantages to the use of AAV for gene transfer. The host range of AAV is broad. Moreover, unlike retroviruses, AAV can infect both quiescent and dividing cells. In addition, AAV has not been associated with human disease, obviating many of the concerns that have been raised with retrovirus-derived gene transfer vectors.

Standard approaches to the generation of recombinant rAAV vectors have required the coordination of a series of intracellular events: transfection of the host cell with an rAAV vector genome containing a transgene of interest flanked by the AAV ITR sequences, transfection of the host cell by a plasmid encoding the genes for the AAV rep and cap proteins which are required in trans, and infection of the transfected cell with a helper virus to supply the non-AAV helper functions required in trans (Muzyczka, N., Curr. Top. Micro. Immunol. 158:97-129, 1992). The adenoviral (or other helper virus) proteins activate transcription of the AAV rep gene, and the rep proteins then activate transcription of the AAV cap genes. The cap proteins then utilize the ITR sequences to package the rAAV genome into a rAAV viral particle. Therefore, the efficiency of packaging is determined, in part, by the availability of adequate amounts of the structural proteins, as well as the accessibility of any cis-acting packaging sequences required in the rAAV vector genome.

Retrovirus vectors are a common tool for gene delivery (Miller, Nature (1992) 357:455-460). The ability of retrovirus vectors to deliver an unrearranged, single copy gene into a broad range of rodent, primate and human somatic cells makes retroviral vectors well suited for transferring genes to a cell.

Retroviruses are RNA viruses wherein the viral genome is RNA. When a host cell is infected with a retrovirus, the genomic RNA is reverse transcribed into a DNA intermediate which is integrated very efficiently into the chromosomal DNA of infected cells. This integrated DNA intermediate is referred to as a provirus. Transcription of the provirus and assembly into infectious virus occurs in the presence of an appropriate helper virus or in a cell line containing appropriate sequences enabling encapsidation without coincident production of a contaminating helper virus. A helper virus is not required for the production of the recombinant retrovirus if the sequences for encapsidation are provided by co-transfection with appropriate vectors.

The retroviral genome and the proviral DNA have three genes: the gag, the pol, and the env, which are flanked by two long terminal repeat (LTR) sequences. The gag gene encodes the internal structural (matrix, capsid, and nucleocapsid) proteins; the pol gene encodes the RNA-directed DNA polymerase (reverse transcriptase) and the env gene encodes viral envelope glycoproteins. The 5′ and 3′ LTRs serve to promote transcription and polyadenylation of the virion RNAs. The LTR contains all other cis-acting sequences necessary for viral replication. Lentiviruses have additional genes including vit, vpr, tat, rev, vpu, nef, and vpx (in HIV-1, HIV-2 and/or SIV). Adjacent to the 5′ LTR are sequences necessary for reverse transcription of the genome (the tRNA primer binding site) and for efficient encapsidation of viral RNA into particles (the Psi site). If the sequences necessary for encapsidation (or packaging of retroviral RNA into infectious virions) are missing from the viral genome, the result is a cis defect which prevents encapsidation of genomic RNA. However, the resulting mutant is still capable of directing the synthesis of all virion proteins.

Lentiviruses are complex retroviruses which, in addition to the common retroviral genes gag, pol and env, contain other genes with regulatory or structural function. The higher complexity enables the lentivirus to modulate the life cycle thereof, as in the course of latent infection. A typical lentivirus is the human immunodeficiency virus (HIV), the etiologic agent of AIDS. In vivo, HIV can infect terminally differentiated cells that rarely divide, such as lymphocytes and macrophages. In vitro, HIV can infect primary cultures of monocyte-derived macrophages (MDM) as well as HeLa-Cd4 or T lymphoid cells arrested in the cell cycle by treatment with aphidicolin or gamma irradiation. Infection of cells is dependent on the active nuclear import of HIV preintegration complexes through the nuclear pores of the target cells. That occurs by the interaction of multiple, partly redundant, molecular determinants in the complex with the nuclear import machinery of the target cell. Identified determinants include a functional nuclear localization signal (NLS) in the gag matrix (MA) protein, the karyophilic virion-associated protein, vpr, and a C-terminal phosphotyrosine residue in the gag MA protein. The use of retroviruses for gene therapy is described, for example, in U.S. Pat. No. 6,013,516; and U.S. Pat. No. 5,994,136, the disclosures of which are hereby incorporated herein by reference.

Additional information on viruses which may be applicable to the methods of the present invention can be found in many virology textbooks (Friedman, Theodore; The Development of Human Gene Therapy, Cold Spring Harbor Laboratory Press, 1998).

The viral vectors administered by the method of the instant invention comprise expression cassettes comprising regulatory elements, such as promoters and enhancers, operably linked to a transgene of choice. Suitable promoters and enhancers are widely available in the art for use in the viral vector of choice. In preferred embodiments, the regulatory elements comprise combinations of promoter and enhancer elements that are able to direct transgene expression preferentially in liver such as those described in PCT US 00/31444, the disclosure of which is incorporated herein by reference. They may comprise combinations of a constitutive or high-expressing promoter and one or more liver-specific enhancer elements.

The strong constitutive promoter may be selected from the group comprising a CMV promoter, a truncated CMV promoter, human serum albumin promoter, and an α-1-antitrypsin promoter. In other embodiments, the promoter is a truncated CMV promoter from which binding sites for known transcriptional repressors have been deleted. The liver-specific enhancer elements may be selected from the group consisting of human serum albumin [HSA] enhancers, human prothrombin [HPrT] enhancers, α-1-microglobulin enhancers and intronic aldolase enhancers. One or more of these liver-specific enhancer elements may be used in combination with the promoter. In one preferred embodiment of an expression cassette, one or more HSA enhancers are used in combination with a promoter selected from the group consisting of a CMV promoter or an HSA promoter. In another preferred embodiment, one or more enhancer elements selected from the group consisting of human prothrombin (HPrT) enhancers and α-1-microglobulin (A1MB) enhancers are used in combination with the CMV promoter. In yet another preferred embodiment, the enhancer elements are selected from the group consisting of HPrT enhancers and A1MB enhancers, and are used in combination with the α-1-antitrypsin promoter.

The present invention provides a method for delivering a viral gene therapy agent to a selected organ of a mammalian subject in order to express a protein encoded by the viral gene therapy agent. The method comprises the steps of placing one or more catheters within the venous vasculature which drains the organ or section of the organ; at least one of the catheters having one or more inflatably expandable members; isolating the organ or section of the organ by occluding flow of fluids within the venous vasculature which drains the organs or section of the organ by inflating one or more of the inflatably expandable members; delivering a viral gene therapy agent with a volume which causes a rise in venous vascular pressure of no more than 40% above the normal venous pressure in the isolated organ or isolated section of the organ; and allowing the gene therapy agent to persist within the isolated section of the vasculature for a period of time sufficient for transfection of a therapeutically effective amount of the agent. It also optionally comprises an additional step of flushing the venous vasculature to remove anti-viral antibodies prior to delivery of the viral gene therapy agent.

In the method of the invention, a viral gene therapy agent is delivered at a volume large enough to perfuse the venous vasculature which drains an isolated organ or isolated section of an organ without elevating the pressure within the said venous vasculature significantly above the normal venous pressure of the isolated organ or isolated section of the organ. In the case of the liver, the normal venous pressure within the isolated organ or isolated section of the organ is measured by the wedged hepatic venous pressure (WHVP). The WHVP is determined by inserting a balloon catheter into the hepatic vein and inflating the balloon catheter to occlude the hepatic vein. The pressure in the occluded vein is measured by a pressure transducer on the tip of the distal end of the balloon catheter, and this measured pressure equals the WHVP. Typical values for the WHVP in human subjects are 40 to 140 mm saline. Higher values may be present in patients with liver, vascular or heart disease.

In the method of the invention, a viral gene therapy agent is delivered to the venous vasculature which drains an isolated organ or isolated section of an organ. In the case of delivery to an entire organ, it would be obvious to one ordinarily skilled in the art that the venous vasculature draining the organ refers to the venous vasculature draining the entire organ. In the case of the liver, the venous vasculature draining the liver refers to the hepatic vein, right or left hepatic veins, sublobar veins, central veins, and sinusoids. The portal veins are not considered part of the venous vasculature, which drains the liver. Furthermore, in the case of the liver, a section of the venous vasculature draining the organ may be occluded according to the method of the invention to isolate a section of the organ. One ordinarily skilled in the art will recognize that the section of the venous vasculature draining the organ and section of the organ isolated according to the methods of the invention may be identified by injecting radiographic contrast agent using one of the catheters, which are used to deliver the viral therapeutic agent. By injecting a volume of contrast agent equal to the volume of viral therapeutic that may be used for the same isolated organ or isolated section of the organ, the isolated section would be identifiable by fluoroscopy. In the case of the liver, contrast agent injected into an occluded hepatic vein or occluded division of a hepatic vein would demonstrate, under fluoroscopy, the section of the liver which was to be isolated.

For the purpose of the present invention, an injection volume may be chosen which, when injected into the isolated organ or isolated section of the organ, causes the venous pressure of the isolated organ or isolated section of the organ to rise by 10%, 20%, 30%, or 40% above the normal venous pressure in the isolated organ or isolated section of the organ. Again, in the case of the liver, the normal venous pressure of the venous vasculature draining the liver would be measured by the WHVP. For a WHVP of 100 mm saline, an injection volume may be chosen to cause the WHVP to rise by no more than 10 mm saline (0.75 mm Hg), no more than 20 mm saline (1.5 mm Hg), no more than 30 mm saline (2.25 mm Hg), or no more than 40 mm saline (3.0 mm Hg). The injection volume may be chosen to equal 1-5%, 5-10%, 10-20%, 20-30%, or 30-40% of the volume of the target organ or portion of the target organ to be treated.

In the method of the invention, the viral gene therapy agent may be delivered to a mammalian subject, such as a human, with pre-existing immunity to the virus. Pre-existing immunity may be recognized by the presence of anti-bodies to a portion of the viral therapeutic agent in the serum of the subject or by identifying a cellular immune response to the viral gene therapy agent. The anti-bodies may be directed towards proteins contained on or within the virus or to the DNA or RNA contained within the virus. A variety of methods exist for identifying anti-bodies within the serum of a subject including enzyme-linked immunosorbent assays (ELISA), radio-immuno assays (RIA), or agglutination assays. Methods for identifying a cellular immune response include mixed-lymphocyte reactions and cell-mediated lympholysis assays. These methods for identifying anti-bodies or measuring cellular immune responses are described in general immunology textbooks (Kuby, Janis; Immunology, 3^{rd} Edition, 1997; Roitt et al., Immunology, 6^{th} ed., 2001, Mosby).

In the method of the present invention, a viral gene therapy agent is delivered to the isolated venous vasculature which drains an organ or section of an organ in order to express a protein encoded by the viral gene therapy agent in the target cells of the organ. Since organs are made up of a variety of cell types, this invention provides a method for maximizing the ratio of expression in target parenchymal cells to expression in non-target cells. Cells which express the virally encoded protein are defined as those cells which have been transfected by the viral therapeutic agent by the method of the invention and subsequently produce a protein encoded by the viral therapeutic agent.

In the case of the liver, target parenchymal cells are hepatocytes, and non-target cells are non-hepatocytes. Non-hepatocytes include vascular endothelial cells, Kupffer cells and supporting stromal cells. Therefore, in the method of the invention, the ratio of hepatocytes expressing protein encoded by the viral gene therapy agent to the ratio of non-hepatocytes expressing protein encoded by the viral gene therapy agent will by maximized. This ratio may be at least 0.2, at least 0.3, at least 0.4, at least 0.5, at least 0.6, at least 0.7, at least 0.8, or at least 0.9.

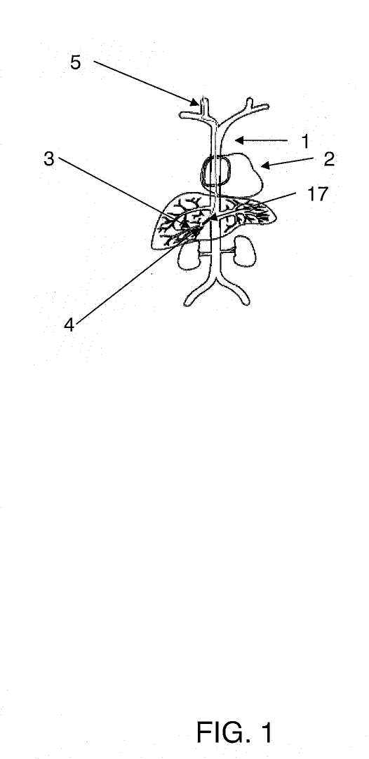

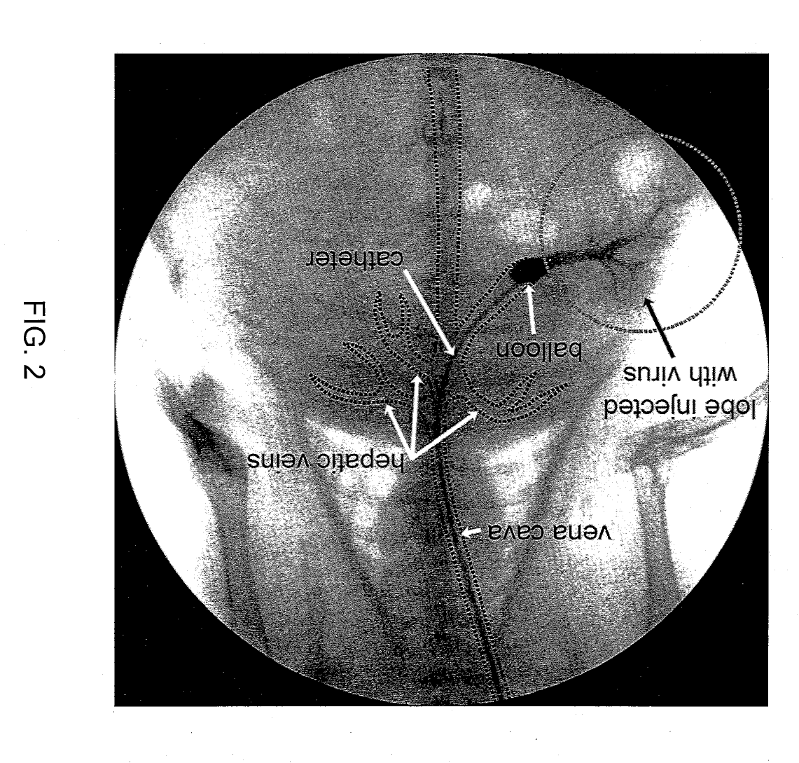

In one embodiment of the present invention, a single lobe of the liver is transfected using a small volume, viral gene therapy agent. A balloon occlusion catheter (4) is inserted through a jugular vein (5), advanced through the superior vena cava (1) and into the desired hepatic vein (3), as depicted in FIG. 1. Immediately prior to endovascular, transcatheter injection of the transfection agent, a balloon (17) on the catheter is inflated to block venous outflow, thus confining the injected solution to the parenchyma of the isolated target lobe. One ordinary skilled in the art would recognize that the same procedure may be performed by accessing the venous vasculature through a number of anatomic locations other than a jugular vein. For example, a femoral vein may also be used.

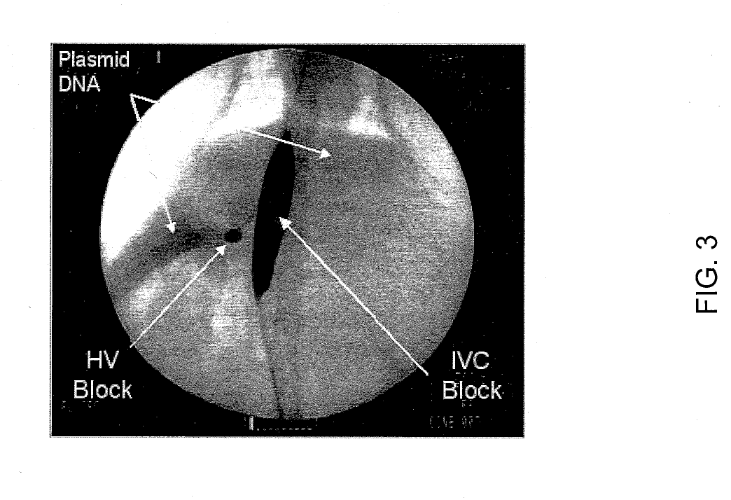

In another embodiment of the invention, a single hepatic lobe is transfected. A balloon occlusion (4) catheter is placed within the lumen of a selected hepatic vein. A second occluding balloon (6) is placed in the hepatic portion of the inferior vena cava to block hepatic venous outflow, as depicted in FIG. 3. Before transcatheter injection of the transfection agent, the balloons (4, 6) are inflated within the inferior vena cava and hepatic vein to block venous outflow, thus confining the injected solution to the parenchyma of the isolated target lobe.

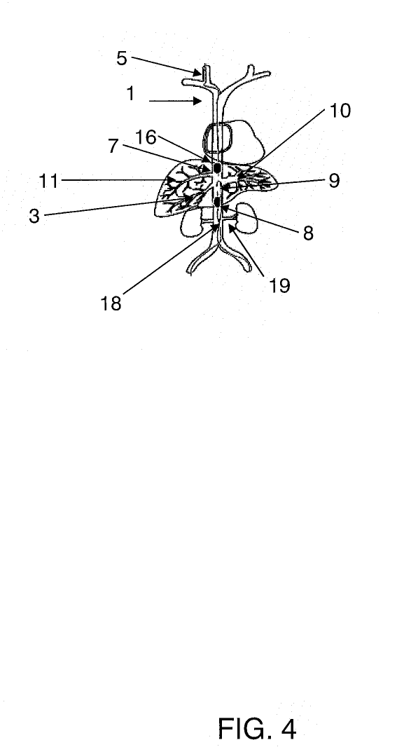

In still another embodiment of the invention, the transfection agent is delivered to the entire liver with a single injection. As depicted in FIG. 4, the liver is isolated through the use of two separate dual-lumen, balloon catheters (7, 8), which are inflated in the inferior vena cava (18) both superior and inferior to the hepatic venous outflow. The transfection agent is then injected through an endovascular catheter (9) positioned between the balloons and flows in a retrograde fashion through the hepatic veins to the entire hepatic parenchyma. As depicted in FIG. 4, the endovascular catheter (9) may be incorporated with one of the balloon occlusion catheters (15), reducing the number of catheters that must be deployed. In all embodiments of the invention, the balloons are sized to each patient's vessels to assure atraumatic blockage of target-organ vascular outflow during the procedure. The methods of the present invention involve the use of a viral gene therapy agent in the course of gene therapy, however, the methods apply equally well to therapeutic injections of chemotherapeutic or other pharmaceutical agents, stem cells, or imaging contrast materials where targeted delivery of a diagnostic or therapeutic solution at controlled pressure to an isolated organ is desired.

The methods of the present invention may also include a “flushing” step prior to viral administration. Flushing utilizes a physiologically appropriate solution, such as saline, to perfuse or partially perfuse the isolated organ or section of the organ prior to administration of virus. Without being limited as to theory, the flushing solution dilutes the blood and physiological fluids of the organ so as to minimize potential interactions between the fluid components and the virus. By minimizing these interactions, overall organ transfection is increased. In a preferred embodiment, when the liver is the target organ, the flushing step prior to viral administration may increase the number and proportion of hepatocytes transfected. In another preferred embodiment, the flushing step prior to viral administration increases the number and proportion of hepatocytes transfected in an animal with pre-existing immunity to the viral vector administered.

The methods of the present invention may also include a variation of the dwell time of the viral gene therapy agent. The dwell time is the time after which the viral gene therapy agent is injected into the target organ and before the occluding balloon is deflated, thereby restoring normal blood flow through the target organ. The dwell time may be minimal, wherein the viral gene therapy agent is injected then immediately recovered. The dwell time may be extended, where the viral gene therapy agent is allowed to dwell within the target organ for a period of time that will not increase the acute toxicity associated with the procedure beyond Grade 2 toxicity. This period of time will depend on the particular target organ at issue and may be determined readily by one in the art. An extended dwell time may be at least two minutes, at least three minutes, at least four minutes, at least five minutes, or longer. The ability to have a dwell time is a feature of the instant invention that is possible due to the retrograde nature of the delivery procedure. Since the method delivers vector via retrograde flow, the target organ may be occluded in order to allow the virus to remain in contact with the organ tissue. Such a dwell time is not generally possible with the anterograde delivery methods used in the prior art. In these anterograde methods, normal blood flow may not be occluded safely.

Immunosuppressive agents may also be administered to animals prior to and following dosing with viral gene therapy vectors in order to minimize or reduce the possibility of immune responses against, for example, either the viral vector or the transgene product. For example, agents may be administered that suppress cytotoxic lymphocytes, which may recognize any expressed viral capsid proteins and may thus eliminate the transduced cells. Immunosuppressive agents that are commonly utilized in the field of organ transplantation are likely immunosuppressive agents suitable for use in combination with the instant methods. Such agents may be used alone or in combination with other such agents. These immunosuppressive agents may include those used for induction and/or those used for maintenance. Exemplary agents include cyclosporine (Neoral®, Sandimmune®), prednisone (Novo Prednisone®, Apo Prednisone®), azathioprine (Imuran®), tacrolimus or FK506 (Prograf®), mycophenolate mofetil (CellCept®), sirolimus (Rapamune®), OKT3 (Muromorab CO3®, Orthoclone®), ATGAM & Thymoglobulin. However, any clinically approved agent that effectuates immunosuppression may be used. Effective immunosuppressive regimes are routinely practiced in the art. Therefore, the appropriate regime and dosing will depend on the particular target organ at issue and may be determined readily by one in the art.

The methods of the present invention may also include the combination of various particular embodiments. For example, flushing of the organ prior to viral delivery may be used in combination with an extended dwell time. Or, flushing of the organ prior to viral delivery may be used in combination with an immunosuppressive regime. Or, flushing of the organ prior to viral delivery may be combined with the use of a serotype selected as having an enhanced tropism for the mammal to be treated. Or, flushing of the organ prior to viral delivery may be used in combination with an extended dwell time and an immunosuppressive regime. The stated examples are intended to illustrate, but not limit, the present invention.

Transgenes encoding for molecules useful when present in the target organ or when secreted into the bloodstream are suitable for use in the instant methods. Such molecules may include proteins and hormones. Exemplary proteins include those deficient in lysosomal storage disorders listed below:

TABLE 1

| Lysosomal storage disease | Defective enzyme |
|---|---|
| Aspartylglucosaminuria | Aspartylglucosaminidase |
| Fabry | α-Galactosidase A |
| Infantile Batten Disease* (CNL1) | Palmitoyl Protein Thioesterase |
| Classic Late Infantile Batten | Tripeptidyl Peptidase |
| Disease* (CNL2) | |
| Juvenile Batten Disease* (CNL3) | Lysosomal Transmembrane Protein |
| Batten, other forms* (CNL4-CNL8) | Multiple gene products |
| Cystinosis | Cysteine transporter |
| Farber | Acid ceramidase |
| Fucosidosis | Acid α-L-fucosidase |
| Galactosidosialidosis | Protective protein/cathepsin A |
| Gaucher types 1, 2*, and 3* | Acid β-glucosidase, or |
| | glucocerebrosidase |
| G_{M1} gangliosidosis* | Acid β-galactosidase |
| Hunter* | Iduronate-2-sulfatase |
| Hurler-Scheie* | α-L-Iduronidase |
| Krabbe* | Galactocerebrosidase |
| α-Mannosidosis* | Acid α-mannosidase |
| β-Mannosidosis* | Acid β-mannosidase |
| Maroteaux-Lamy | Arylsulfatase B |
| Metachromatic leukodystrophy* | Arylsulfatase A |
| Morquio A | N-Acetylgalactosamine-6-sulfate |
| | sulfatase |
| Morquio B | Acid β-galactosidase |
| Mucolipidosis II/III* | N-Acetylglucosamine- |
| | 1-phosphotransferase |
| Niemann-Pick A*, B | Acid sphingomyelinase |
| Niemann-Pick C* | NPC-1 |
| Pompe* | Acid α-glucosidase |
| Sandhoff* | β-Hexosaminidase B |
| Sanfilippo A* | Heparan N-sulfatase |
| Sanfilippo B* | α-N-Acetylglucosaminidase |
| Sanfilippo C* | Acetyl-CoA:α-glucosaminide |
| | N-acetyltransferase |
| Sanfilippo D* | N-Acetylglucosamine-6-sulfate |
| | sulfatase |
| Schindler Disease* | α-N-Acetylgalactosaminidase |
| Schindler-Kanzaki | α-N-Acetylgalactosaminidase |
| Sialidosis | α-Neuramidase |
| Sly* | β-Glucuronidase |
| Tay-Sachs* | β-Hexosaminidase A |
| Wolman* | Acid Lipase |

*CNS involvement

Other exemplary proteins are those to treat other diseases such as Alzheimer's disease in mammals, including humans. In such methods, the transgene encodes a metalloendopeptidase. The metalloendopeptidase can be, for example, the amyloid-beta degrading enzyme neprilysin (EC 3.4.24.11; sequence accession number, e.g., P08473 (SWISS-PROT)), the insulin-degrading enzyme insulysin (EC 3.4.24.56; sequence accession number, e.g., P14735 (SWISS-PROT)), or thimet oligopeptidase (EC 3.4.24.15; sequence accession number, e.g., P52888 (SWISS-PROT)).

Additionally, the transgene may encode a protein selected from the group consisting of insulin growth factor-1 (IGF-1), calbindin D28, parvalbumin, HIF1-alpha, SIRT-2, VEGF, SMN-1, SMN-2, GDNF, and CNTF (Ciliary neurotrophic factor). Such proteins may be suitable for the treatment of Amyotrophic Lateral Sclerosis (ALS) using this method via mediating effects following secretion into the bloodstream. ALS is a progressive, lethal neuromuscular disease that is associated with the degeneration of spinal and brainstem motor neurons. Progression of the disease can lead to atrophy of limb, axial and respiratory muscles. Over expression of superoxide dismutase-1 (SOD1) gene mutations in mice and rats recapitulates the clinical and pathological characteristics of ALS in humans. Compounds active in retarding symptoms in this model have been shown to be predictive for clinical efficacy in patients with ALS, and therefore is a therapeutically relevant model of this disease. Such mouse models have been previously described in Tu et al. (1996) P.N.A.S. 93:3155-3160; Kaspar et al. (2003) Science 301:839-842; Roaul et al. (2005) Nat. Med. 11(4):423-428 and Ralph et al. (2005) Nat. Med. 11(4):429-433.

In another example, the transgene may encode a protein deficient in hemophilia, such as Factor VIII or Factor IX. The stated examples are intended to illustrate, but not limit, the present invention.

The following representative examples are intended to illustrate, but not limit, the present invention. While the representative procedures are performed in rabbits, they are successfully performed within parameters clinically feasible in other mammals such as non-human primates and human subjects.

### EXAMPLE 1

### Catheter Based Delivery of an Adenoviral Gene Therapy Vector to the Rabbit Liver

For each of the experiments, New Zealand white rabbits weighing approximately 4 kg each were used (Millbrook Farms, Amherst, Mass.). The adenoviral vector utilized, Ad2βgal, has a serotype 2 backbone and is deleted of E1 but retains the E3 and E4 regions. The expression cassette consists of a cytomegalovirus (CMV) immediate-early promoter and enhancer, the cDNA for a nuclear-localized β-galactosidase, and an SV40 polyadenylation signal (Armentano, D., et al. (1997). J. Virol. 71:2408-2416). Each rabbit was injected with 1.5×10¹² viral particles/kg (particle:infectious unit ratio=10:1) of the Ad2βgal virus.

Adenoviral gene therapy vector was delivered to the liver of rabbits utilizing an embodiment of the method of the present invention as follows. To access the vascular system of the rabbit, the jugular vein was exposed via a midline incision beginning at the mandibular arch and extending caudally followed by blunt dissection of the muscle tissue that exposed the right external jugular vein. An angiocatheter needle was inserted into the exposed jugular and a guidewire was inserted into the needle. Under fluoroscopic guidance, the guidewire was advanced through the superior vena cava and heart and into the hepatic venous circulation. The angiocatheter needle was removed and a balloon occlusion catheter was placed over the guidewire and inserted into the jugular vein. The catheter was advanced along the guidewire into the hepatic vein under fluoroscopic guidance. The guidewire was removed and a small amount of non-ionic contrast agent in saline was injected to confirm proper catheter positioning. The occlusion balloon was then inflated with contrast media and its position was again confirmed by injection of a small volume of contrast media. Inflation of the occlusion balloon within the hepatic vein blocked the hepatic vein draining the right lobe of the liver, as depicted in FIG. 1. Viral solution was then injected retrograde into the selected lobe of the liver at a rate of approximately 1 ml/sec, which was followed by injection of a small volume (1 ml) of PBS to wash any virus remaining in the catheter system into the lobe. Virus was allowed to dwell in the tissue for approximately one minute during which backflow into the injection syringe was prevented. Following the dwell time, a volume representing the injectate volume was recovered by pulling back on the syringe at a rate approximately equal to the injection rate. The occlusion balloon was then deflated and the catheter removed. Hemostasis was achieved and the incision was closed using the appropriate materials.

Using the above delivery method, several bolus volumes (3, 8, and 20 ml) containing the same total number of viral particles (1.5×10¹² viral particles/kg of Ad2βgal vector) were evaluated in the rabbit to measure the relative hepatocellular transduction mediated by the adenoviral vector at each volume. An average lobe of the rabbit liver was estimated to be approximately 20 g, which was used to set an upper limit of 20 ml for the delivered volume of the viral bolus. This upper limit was anticipated to distribute the viral bolus throughout the injected lobe. Based on this assumption, smaller volumes of 3 ml and 8 ml were also chosen to achieve less than complete distribution of the viral bolus throughout the injected lobe. Three days post-treatment, the rabbits were sacrificed. Beta-galactosidase expression was measured in the liver, kidney, lung, and spleen.

Expression was characterized by using AMPGD (chemiluminogenic substrate for β-galactosidase, 3-{4-Methoxyspiro[1,2-d]oxetane-3,29-tricyclo[3.3.1.13,7)decan]-yl}phenyl-β-D-galactopyranoside) using a luminescence-based assay to obtain relative expression levels. In the luminescence assay, 100 to 200 mg of tissue was homogenized in 2× volume of 1× lysis buffer (Tropix Galacto-Light Plus Kit, Tropix) using a Janke and Kunkel Ultra-Turrax T25 homogenizer. The homogenate was subjected to two rounds of freeze and thaw, followed by heat inactivation of endogenous β-galactosidase in a 48° C. water bath for 1 hour. Samples were centrifuged at 14,000 rpm for 10 min at 4° C., and the supernatants transferred to a clean 1.5 ml Eppendorf tube. The protein concentration of each sample was assayed using the Micro BCA Protein Assay Reagent Kit (Pierce), and the absorbance at 570 nm read using BioRad EIA plate reader. Beta-galactosidase activity of each sample was assayed using the Tropix Galacto-Light Plus Kit per the manufacturer's instructions, and read using the Tropix TR717Microplate Luminometer, and WinGlow software.

Immunohistochemistry on tissue samples was generally performed as follows. Four millimeter slices of tissue were fixed in formalin-zinc overnight, rinsed in PBS, embedded in paraffin and sectioned. Sections were deparaffinized by successive washes in Hemo-D, 100%, 95%, 70%, and 50% ethanol, double distilled water, and PBS. Endogenous peroxide activity was eliminated with a 3% solution of hydrogen peroxide in methanol, followed by rehydration in water. Sections were blocked in 5% goat serum in PBS. The sections were incubated with mouse anti-beta-galactosidase overnight at 4° C., washed twice in PBS, followed by incubation with affinity purified, peroxidase labeled goat anti-mouse IgG for one hour at 37° C., followed by two washes in PBS, and one wash in 0.5 mM Tris-HCl pH 7.5. Peroxidase label was detected using the Liquid DAB Substrate-Chromogen System (DAKO) per the manufacturer's instructions, followed by Methyl Green counterstaining of the section.

Quantitation of cells expressing beta galactosidase was generally performed as follows. Metamorph software was used to distinguish and quantitate the nuclei of hepatocytes and non-hepatocytes infected with Ad2β-gal. Color development times for immunohistochemical detection of beta-galactosidase signal were held constant to ensure consistent MetaMorph detection of infected cells. Each liver section was scanned into Metamorph and five regions (each 1 mm²) were chosen from each liver section for MetaMorph analysis. Color thresholding was used to distinguish the brown DAB (3,3′-Diaminobenzidine) signal representing positive beta-galactosidase nuclei from the blue-green nuclei of non-infected cells. Color thresholding and all subsequent MetaMorph classifications were optimized by preliminary empirical analysis of the nuclei from a sample liver section, followed by human validation of each classification.

To classify nuclei into hepatocytes, non-hepatocytes or unknown objects, the “total area” and “elliptical form factor” of all beta-galactosidase positive nuclei were measured by MetaMorph. The total area (TA) is defined as the sum all the contiguous pixels of a given beta-galactosidase positive nucleus that meets the color threshold. The elliptical form factor (EFF) is defined as the length of a given beta-galactosidase positive nucleus divided by its breadth. For example, the EFF of a perfect circle is 1.0, while the EFF of an ellipse is generally greater than 1.25.

The nuclei of single hepatocytes are roughly spherical and produce an EFF≦1.25 and a TA=16-18 pixels. The nuclei of double hepatocytes appear as two closely spaced spheres that histologically cannot be dissected by MetaMorph, and produce an EFF that is generally greater than 1.5, with a TA>18 pixels. The beta-galactosidase positive nuclei of non-hepatocytes are predominantly derived from the liver macrophages (Kupffer cells) and endothelial cells, and produce an EFF>1.25 and a TA>6 and ≦18.

Due to the position and orientation of a given nucleus within the sectional plane, some beta-galactosidase positive nuclei were classified as unknown objects because they could not be accurately classified into hepatocytes or non-hepatocytes. Unknown objects were assumed to represent hepatocytes and non-hepatocytes in proportion to their respective populations, and were not factored into any quantitative analysis. Unknown objects had a TA equal to 1 but ≦6, and any value for EFF, or a TA>6 but <16 and an EFF≦1.25. Five fields (each 1 mm²) from each proximal, medial and distal section from each of the four main lobes of the rabbit liver were collected for MetaMorph analysis.

In the rabbits treated with the 3 ml bolus injection, beta-galactosidase expression in the injected liver lobe ranged from 20-300×10⁶ RLU/mg tissue with relatively more expression distal to the injection site. Expression in the un-injected liver lobes ranged from 1-12×10⁶ RLU/mg tissue with no consistent proximal-distal expression pattern. Expression in the kidney, lung, and spleen was largely undetectable. Immunohistochemical localization of beta-galactosidase expressing cells in the injected liver lobe demonstrated that the cellular anatomic distribution of expression was largely confined to areas surrounding the central vein, which was the delivery route for the viral bolus. The majority of these expressing cells were hepatocytes. In the non-injected liver lobes, beta-galactosidase expressing cells were immunolocalized largely to areas surrounding the portal triad.

Beta-galactosidase expression in the livers of rabbits treated with the 8 ml bolus injection was significantly higher than that obtained with the 3 ml bolus injection. Beta-galactosidase expression in the injected liver lobe ranged from 300-600×10⁶ RLU/mg tissue while expression in the un-injected liver lobes ranged from 100-300×10⁶ RLU/mg tissue. As noted with the 3 ml injection bolus, expression increased in the injected liver lobe in a proximal to distal direction relative to the injection site. In the un-injected lobe, there with no consistent proximal-distal expression pattern. Expression in the kidney, lung, and spleen was largely undetectable. Immunohistochemical localization of beta-galactosidase expressing cells in the injected liver lobe demonstrated an essentially uniform pattern of expression throughout the lobe; the majority of these expressing cells were hepatocytes. Beta-galactosidase expression was also uniform in the un-injected lobes with respect to liver architecture as measured by immunohistochemical localization.

In the rabbits treated with the 20 ml bolus injection, beta-galactosidase expression in the injected liver lobe was approximately half that achieved with the 8 ml volume with an analogous proximal to distal gradient in expression. Beta-galactosidase expression in the non-injected liver lobes was uniformly distributed and was approximately 10-fold lower than expression measured in the injected lobe. Immunohistochemical localization of beta-galactosidase expressing cells in the injected liver lobe demonstrated an essentially uniform pattern of expression throughout the lobe; the majority of these expressing cells were hepatocytes. Expression in the kidney, lung, and spleen was largely undetectable.

FIG. 6 illustrates the distribution of expression found using 3, 8, and 20 ml injection volumes. Immunohistochemically, the 8 ml volume appeared to result in the greatest number of expressing cells, both in the injected lobe (Lobe 1) (FIG. 6D) and in the un-injected lobe (Lobe 4) (FIG. 6E). These qualitative results were confirmed by quantitative determinations of β-galactosidase protein levels in 3 tissue cores removed from each lobe. FIGS. 6C, 6F, and 6I schematically show the average expression levels determined from three locations (proximal, medial, and distal to the entry of the hepatic vein from the vena cava) in each of the four major lobes. Summation of the values for β-galactosidase expression from each of the four lobes demonstrated that the 8 ml injection volume was ˜40% higher than the 3 ml injection volume and ˜60% higher than the 20 ml injection volume. From these analyses, the 8 ml volume was chosen and all subsequent experiments, both local (catheter) and systemic injections, used an 8 ml injection volume.

In addition to total expression, FIG. 6 also illustrates different distributions of expression that resulted from the different injection volumes. Thus, for example, with a 3 ml injection volume, expressing cells in the injected lobe (FIG. 6A) were largely confined to regions surrounding the hepatic vein, with relatively many fewer expressing cells localized around the portal triad or in the un-injected lobe (FIG. 6B). Together with the quantitative results, which showed less expression in the un-injected lobes (FIG. 6C), demonstrate that the initial distribution of virus was confined to the immediate regions surrounding the injected hepatic vein.

In contrast to the results obtained with a 3 ml injection volume, FIG. 6 also demonstrates that the 8 ml injection volume achieved a significantly greater distribution of the viral bolus. Thus, FIG. 6D shows expressing cells throughout the liver acinus, and not restricted to regions around the hepatic vein, and FIG. 6E shows that some of the injected virus has infected the un-injected lobe. These results are confirmed and quantified in FIG. 6F, which demonstrates significant expression in both injected (Lobe 1) and un-injected lobes (Lobes 2, 3, and 4). These data are consistent with an initial distribution of the viral bolus throughout the injected lobe and into the portal and venous circulation of the un-injected lobes that could subsequently redistribute and infect the un-injected lobes.

The 20 ml injection volume, which was expected to distribute the initial viral bolus well beyond the injected lobe, resulted in widespread but fewer (compared to the 8 ml volume) expressing cells in both the injected (FIG. 6G) and un-injected (FIG. 6H) lobes. These qualitative results were confirmed by β-galactosidase quantitation (FIG. 6I), and are consistent with a scenario in which the injected viral bolus was distributed well into the portal circulation.

### EXAMPLE 2

### Comparison of Local Vs. Systemic Administration in Naive Rabbits

To ask whether local delivery of a viral vector conferred an advantage over systemic delivery, Ad2βgal virus was delivered to rabbits via either 1) local delivery to the liver using the balloon catheter-mediated delivery described in Example 1 or 2) via systemic delivery using intravenous injection. Independent of delivery route, each rabbit was injected with 1.5×10¹² viral particles/kg of the Ad2βgal virus.

Systemic delivery of the viral vector was carried out according to the following protocol. The marginal ear vein of a sedated rabbit was accessed using a 20-gauge angiocatheter needle secured to the ear with rolled gauze and medical tape. A luer-lock flush was attached to the catheter, and Benadryl; 1 mg/kg, IV was administered to control possible anaphylactic responses. An 8 ml volume of saline containing the Ad2βgal virus was injected into the ear vein at a rate of approximately 1 ml/sec. Localized delivery of the Ad2βgal virus to the liver was performed using the balloon catheter-mediated delivery described in Example 1.

Toxicity of the various procedures was evaluated as follows. Blood was collected from each rabbit just prior to virus administration, and one, two and three days following virus administration. Cell count differentials (white and red blood cells, hemoglobin, hematocrit, mean corpuscular volume, mean corpuscular hemoglobin concentration, nucleated red blood cells, segmented heterophils (rabbit neutrophils), lymphocytes, monocytes, eosinophils, basophils, platelets), and serum chemistry profiles (alkaline phosphatase, alanine aminotransferase, aspartate aminotransferase, creatine kinase, albumin, total protein, globulin, total and direct bilirubin, BUN, creatine, cholesterol, glucose, calcium, phosphorus, bicarbonate, chloride, potassium, and sodium) were performed.

The livers of treated rabbits were analyzed for beta-galactosidase expression. Bacterial beta-galactosidase expression in rabbit liver homogenates was quantified as described in Example 1 or was quantified using a commercially available ELISA kit (Roche) per the manufacturer's instructions. Immunohistochemistry on tissue samples was performed as described in Example 1. Morphologic analysis was performed to determine the expression pattern in liver, and the transfection ratio of liver hepatocytes to liver non-hepatocytes was determined.

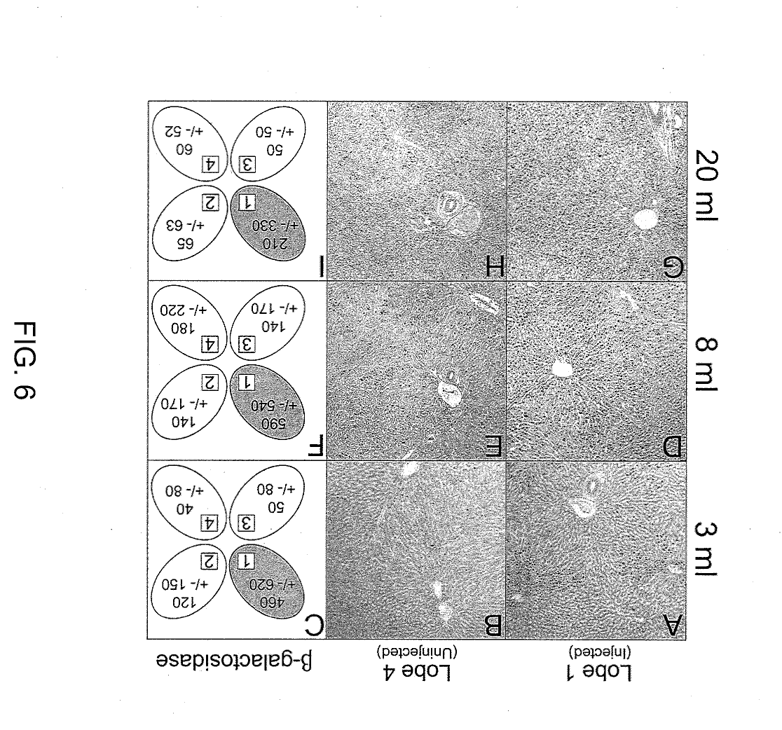

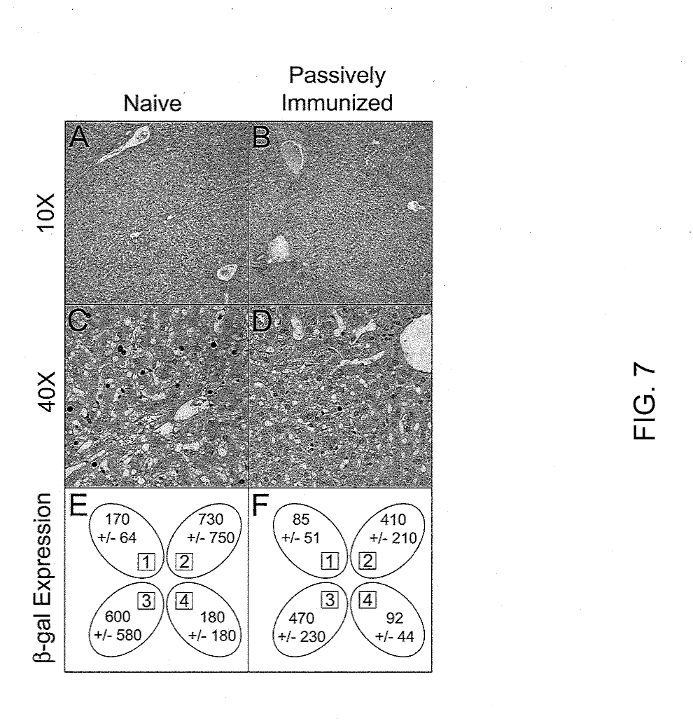

FIGS. 7A and 7C shows that systemic administration of Ad2βgal in these animals resulted in a relatively uniform distribution of expression with respect to liver architecture and that the majority of the transduced cells appeared to be hepatocytes, as indicated by the nuclear β-galactosidase staining highlighting their circular nuclei. This uniformity of transduced cells was essentially the same across all liver lobes (data not shown), and the quantitative data shown in FIG. 7E confirms this assessment, viz., all lobes show approximately equal β-galactosidase expression as determined by ELISA.

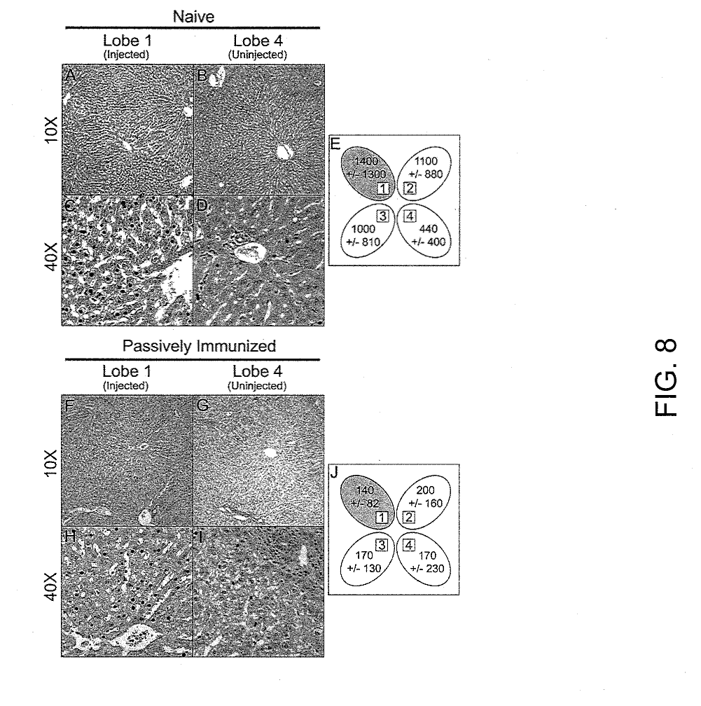

Using the localized delivery method of the identical viral dose and injection volume (8 ml), FIG. 8A demonstrates that the resulting expressing cells in the injected lobe (Lobe 1) were distributed throughout the acinus, consistent with the earlier volume evaluation study in Example 1 (FIG. 6). At higher magnification (FIG. 8C), the vast majority of cells expressing the nuclear-localized β-galactosidase in the injected lobe appeared to be hepatocytes. As compared to the injected lobe, FIG. 6B shows that the relative number of expressing cells in the un-injected lobe (Lobe 4), was significantly less. As in the injected lobe, expressing cells in the un-injected lobe appeared to be mostly hepatocytes, and were uniformly distributed within the acinus.

These qualitative findings in naive animals were supported by the quantitative determination of β-galactosidase depicted schematically in FIG. 6E, which showed significantly greater overall expression in the injected lobe compared to the un-injected lobes. As seen in the volume evaluation experiments (FIG. 6), all un-injected lobes were transduced to approximately the same extent.

FIG. 9A demonstrates that local, catheter mediated delivery using balloon catheters via a hepatic vein route in naive rabbits resulted in ˜2 to 3-fold more infected cells in the injected lobe than in the non-injected lobe; the proportion of expressing cells that could be identified as hepatocytes in both the injected and un-injected lobes was essentially the same (˜0.7) in both injected and non-injected lobes.

FIG. 9D demonstrates that systemic delivery in naive animals results in an overall 2 to 3-fold decrease in total expressing cells when compared to local delivery (FIG. 9A). Evaluation of both Lobes 1 and 4 (note that there are no “injected” or “un-injected” lobes with a systemic delivery), gave essentially identical numbers of expressing cells. Evaluation of the hepatocyte fraction of expressing cells was essentially identical in the injected (0.63) and un-injected (0.69) lobes, and was roughly equivalent to that obtained after local delivery (˜0.71; FIG. 9A).

Taken together, the data demonstrate that local, catheter-mediated delivery of adenoviral vector confers an advantage relative to systemic delivery. Local delivery using a hepatic vein approach resulted in 2-3 fold more expression (FIGS. 8E and 9A) compared to delivery using a systemic approach (FIGS. 7E and 9D).

The toxicities that resulted from delivering Ad2-βgal were minor, both by local (balloon-catheter) and systemic approaches. Cell blood count and serum chemistry analyses were performed on blood samples taken from rabbits prior to surgery (baseline), and one, two, and three days following injection of adenovirus, or a sham injection of saline.

A mild, yet statistically significant lymphopenia (50-60% decrease vs. baseline), was apparent within 24 hours in animals treated with virus. A mild but statistically significant (50% decrease vs. baseline) thrombocytopenia was apparent within 24 hours in animals treated with virus. A mild, yet statistically significant heterophilia (100-200% increase vs. baseline) was apparent within 24 hours in animals treated with virus or animals subjected to sham surgery (with a local injection of saline). Statistically significant elevations in serum creatine kinase (100-1000% increase vs. baseline) were apparent within 24 hours in all animals treated with virus or animals subjected to sham surgery (local injection of saline). All blood cell counts and serum chemistry profiles returned to normal within three days following administration of virus or sham surgery.

Histopathological assessment of hematoxylin and eosin stained liver sections from animals infected by local or systemic delivery of virus or uninfected animals, revealed no consistent hepatocellular changes that could be correlated with any specific treatment.

### EXAMPLE 3

### Comparison of Local Vs. Systemic Administration in Passively Immunized Rabbits

Local, catheter-mediated delivery of adenoviral vector was compared to systemic delivery in animals with anti-viral immunity to examine whether local delivery conferred an advantage over systemic delivery in this context. To accomplish this, the experiments in Example 2 were repeated in rabbits passively immunized with pooled human serum of known anti-adenoviral type 2 titers (anti-Ad2.)

One day prior to administration of virus a marginal ear vein of the sedated rabbit was accessed using a 20-gauge angiocatheter needle secured to the ear with rolled gauze and medical tape. A luer-lock flush was attached to the catheter, and Benadryl was administered intravenously (1 mg/kg) to control possible anaphylactic responses. Forty milliliters of pooled human serum (Valley Biomedical, Winchester, Va.) containing anti-Ad2 antibodies was then injected at a rate of 0.1 ml/sec. The pooled human serum had anti-Ad2 titers (total titer=12,800, neutralizing titer=3,200) approximately ten-fold the average human anti-Ad2 titers (unpublished data). Thus, dilution of this delivered dose into the total rabbit circulation (estimated at 350-400 ml) was predicted to give a final anti-Ad2 titer approximating the average human titer. Actual determination of the anti-Ad2 titers at the time of Ad2βgal administration gave a total anti-Ad2 titer of 1,600 and a neutralizing titer of 400 in all animals.

Anti-Ad2 antibody titers in the pooled human serum and the rabbit serum were determined by ELISA. Serial dilutions of serum were added to wells of a 96-well plate coated with heat-inactivated adenovirus type 2. Bound virus-specific antibodies were detected with horseradish peroxidase-conjugated goat anti-rabbit immunoglobulin G, IgM, and IgA. A 30 minute incubation with colorimetric substrate was then used to detect rabbit anti-Ad2 antibodies. Anti-virus titers were defined as the reciprocal of the highest serum dilution that produced an OD490≧0.1.

Adenovirus neutralizing antibody titers in the pooled human serum were assessed by its ability to inhibit transduction of a susceptible cell line by Ad2-βgal. HeLa cells (ATTC) were plated in flat-bottom 96-well tissue culture plates and incubated overnight at 37° C. in a 5% CO₂ atmosphere. Serum samples were serially diluted from 1:25-1:6400 in DMEM and Ad2-βgal (50 MOI) was added to the serum dilutions and incubated for 1 hr at 37° C. before being transferred to the plated cells and continued in culture for 3 days. On the 3rd day, cells were harvested and lysed. Lysates were assayed for substrate conversion using a commercially-available AMPGD/β-galactosidase assay kit. The neutralizing antibody titer of the serum samples was defined as the reciprocal of the dilution giving ≧50% reduction in measured RLUs relative to a negative control (human immunoglobin-depleted serum.) Total anti-Ad2 titers at the time of virus administration were 1:1600 in all rabbits; neutralizing titers were 1:400. These titers are essentially the same as those in a human with an average anti-Ad2 titer.

One day after passive immunization, Ad2βgal virus was delivered via either 1) systemic delivery using intravenous injection or 2) local delivery to the liver using the balloon catheter-mediated delivery as described in Examples 1 and 2. Unless stated, the materials and methods used were similar to those utilized in Examples 1 and 2. Independent of delivery route, each rabbit was injected with 1.5×10¹² viral particles/kg of the Ad2βgal virus.

Systemic administration of Ad2βgal in passively immunized rabbits resulted in uniform β-galactosidase expression with respect to liver architecture, with no apparent concentration of expressing cells around the central vein or portal triad (FIGS. 7B and 7D.) However, compared to systemic administration of an identical dose of virus in a naive animal (FIGS. 7A and 7C), systemic administration in rabbits with pre-existing anti-Ad2 antibody resulted in attenuated expression as total β-galactosidase expression was reduced ˜2 fold (FIG. 7F.)

With respect to the type of cells transfected following passive immunization (hepatocytes vs. non-hepatocytes), FIG. 9E shows that systemic delivery in passively immunized rabbits resulted in a 10-fold decrease in the number of infected hepatocytes (18.0±14 hepatocytes/field) compared to systemic delivery in a naive animal (253±166 hepatocytes/field) (FIG. 9D). However, in the presence of anti-Ad2 antibodies (passively Immunized), systemic delivery resulted in an only nominal decrease in the number of infected non-hepatocytes (124±47 non-hepatocytes/field) (FIG. 9E) compared to systemic delivery in a naive animal (147±82 non-hepatocytes/field) (FIG. 9D). These data are thus consistent with the qualitative and quantitative expression data obtained (FIG. 7), which also show a 2-3 fold decrease in expression due to passive immunity. Therefore, the most dramatic effect of anti-Ad2 antibodies following systemic administration of virus was a decrease in the fraction of infected hepatocytes from 66% of all infected cells in naive animals to 14% of all infected cells in passively immunized animals. Thus, systemic delivery in the presence of anti-vector antibodies led to reduced expression where the majority of expressing cells were non-hepatocytes such as liver sinusoidal endothelial cells and Kupffer cells (FIG. 9E). Only ˜15% of the expressing cells were identified as hepatocytes in passively-immunized animals.

Local delivery of Ad2βgal in passively-immunized animals resulted in a similar distribution of β-galactosidase expressing cells in the injected lobe (FIG. 8F) as that seen in naive animals receiving Ad2βgal (FIG. 8A). Comparisons of local, catheter-mediated administration between naive rabbits (FIG. 8E) and passively-immunized rabbits (FIG. 8J) suggests that the presence of anti-Ad2 antibodies resulted in an approximately 5-10 fold reduction in overall liver β-galactosidase expression.

In addition to the decrease in overall β-galactosidase expression in passively immunized animals, the proportion of cell types expressing β-galactosidase was also altered compared to the proportions in naive animals treated with local, catheter-mediated delivery of adenoviral vector. The proportion of expressing cells that could be identified as hepatocytes in the injected lobe decreased from 0.72 in naive animals to 0.45 in passively immunized animals (FIGS. 9A and 9B.) More striking were the differences in the un-injected lobe, where this ratio decreased from 0.70 to 0.09 due to passive immunization (FIGS. 9A and 9B.)

In summary, overall β-galactosidase expression summed over all regions of the liver in passively immunized animals was essentially the same after local and systemic delivery, as quantified by both ELISA (FIG. 7 and FIG. 8) and Metamorph (FIG. 9) analyses. Importantly, however, the fraction of β-galactosidase expressing cells identified as hepatocytes following local delivery was ˜7 fold greater. This suggests that the predominant portion of the total β-galactosidase expression following systemic delivery in passively immunized animals is derived from non-hepatocytes, and this contention is supported by both the qualitative immunohistochemical (FIGS. 8G and 8I) and the quantitative Metamorph data (FIG. 9E). Also, despite the negative effect anti-Ad2 antibody has on total infection and expression, local delivery is able to preferentially target hepatocytes, the desired target cells for therapeutic gene therapy. Thus, while systemic delivery to immunized rabbits resulted in ˜14% of the expressing cells identified as hepatocytes, local delivery resulted in ˜45% of the expressing cells being hepatocytes in the injected lobe, and ˜10% in the un-injected lobes. The lower percentage of expressing hepatocytes in the non-injected lobes is consistent with a greater degree of interaction of the virus with anti-viral antibodies as it distributes among the non-injected liver lobes.

For passively immunized animals, it is clear from these results that localized delivery provides a significant advantage over systemic delivery. After localized delivery in passively immunized animals, the ratio of expression of the transgene in hepatocytes to non-hepatocytes is significantly higher than the ratio after systemic delivery in passively immunized animals. Therefore, it is clear that this method provides an advantage when administering a viral gene therapy agent to animals with pre-existing immunity.

### EXAMPLE 4

### Catheter Based Delivery of an Adenoviral Gene Therapy Vector to the Rabbit Liver in Passively Immunized Rabbits with Saline Pre-Flushing of the Liver

Local, catheter-mediated delivery of adenoviral vector was evaluated with the addition of a “flushing” step in which 20 ml of saline were infused through the catheter just prior to virus administration. This was performed in animals with anti-Ad2 viral immunity to examine whether pre-flushing of the liver with saline would confer an advantage over delivery without a pre-flushing step in this context.

To accomplish this, rabbits were passively immunized with pooled human serum of known anti-adenoviral type 2 titers (anti-Ad2) as described in Example 3. One day after passive immunization, the balloon catheter-mediated delivery procedure described in Examples 1 and 2 was followed with the addition of the following step. Immediately prior to Ad2βgal delivery, a 20 ml volume of saline was delivered through the catheter.

The addition of the saline flush to the local, catheter-mediated delivery of Ad2βgal in passively-immunized animals increased both the number of cells expressing the transgene and the proportion of hepatocytes expressing β-galactosidase as compared to passively-immunized animals treated with local, catheter-mediated delivery that did not receive a saline flush. In the passively-immunized that received a saline pre-flush, the number of expressing hepatocytes was increased approximately 5-fold when compared to animals that did not receive the pre-flush (FIGS. 9B and 9C). The proportion of expressing cells that could be identified as hepatocytes in the injected lobe also increased, and was 0.68 (FIG. 9C) as compared to 0.45 in passively immunized animals without a saline pre-flush (FIG. 9B) and to 0.71 in non-immunized animals (FIG. 9A) More striking were the differences in the un-injected lobe, where the proportion of expressing cells that could be identified as hepatocytes increased from 0.09 to 0.40 due to the saline pre-flush in passively immunized animals (FIGS. 9B and 9C.) For passively immunized animals, it is clear that saline pre-flushing in addition to the local, catheter-mediated delivery of viral vectors provides a significant advantage in animals with a pre-existing immunity to the viral vector. With the saline pre-flush, both the overall number of expressing cells and the fraction of hepatocytes expressing the transgene is significantly higher than without saline flushing in passively immunized animals. Therefore, it is clear that this embodiment of the instant method provides an advantage when administering a viral gene therapy agent to animals with pre-existing immunity.

### EXAMPLE 5

### Catheter Based Delivery of an Adeno-Associated Virus Gene Therapy Vector to the Rabbit Liver

For each of the experiments, New Zealand white rabbits weighing approximately 4 kg each were used (Millbrook Farms, Amherst, Mass.). The adeno-associated viral vector utilized, AAV2/8DC190HAGAL (AAV2/8), comprises the capsid region of the AAV8 serotype and the inverted terminal repeats of the AAV2 serotype. The expression cassette comprises two α-1 microglobulin enhancers, a α-1-antitrypsin promoter, and the human α-galactosidase A transgene. Rabbits were injected with 1.25×10¹¹ or 1.25×10¹² DNA-ase resistant particles (drp)/kg of the AAV2/8 virus (in a total volume of 8 ml) via local delivery to the liver using the balloon catheter-mediated delivery as described in Examples 1-3. Unless stated, the materials and methods used were similar to those utilized in Examples 1-3.

Human α-galactosidase A (AGAL) expression was measured in the serum of animals using an enzyme-linked immunosorbent assay specific for human α-galactosidase A as previously described [Ziegler et al., (1999). Hum Gene Ther. July 1; 10(10):1667-82.]

In rabbits treated with 1.25×10¹¹ drp/kg of the AAV2/8 virus, AGAL expression in the serum ranged from 3-31 ng AGAL/ml serum. In rabbits treated with 1.25×10¹² DNA-ase resistant particles (drp)/kg of the AAV2/8, AGAL expression in the serum ranged from 15-132 ng AGAL/ml serum.

### EXAMPLE 6

### Time Course of AGAL Expression Following Catheter Based Delivery of an Adeno-Associated Virus Gene Therapy Vector to the Rabbit Liver

New Zealand white rabbits weighing approximately 4 kg each were used (Millbrook Farms, Amherst, Mass.). The adeno-associated viral vector utilized, AAV2DC190HAGAL (AAV2/2), comprises the capsid region and the inverted terminal repeats of the AAV2 serotype. The expression cassette comprises two α-1 microglobulin enhancers, a α-1-antitrypsin promoter, and the human α-galactosidase A transgene. Rabbits were injected with 5×10¹² DNA-ase resistant particles (drp) of the AAV virus in a total volume of 8 ml via local delivery to the liver using the balloon catheter-mediated delivery as described in Examples 1-3. Unless stated, the materials and methods used were similar to those utilized in Examples 1-3.

Human α-galactosidase A (AGAL) expression was measured in the serum of animals using an enzyme-linked immunosorbent assay specific for human α-galactosidase A as previously described [Ziegler et al., (1999). Hum Gene Ther. July 1; 10(10):1667-82.] AGAL expression was measured over a period of 84 days.

Anti-AGAL antibody titers in the serum of treated rabbits were also measured over time using ELISA. Serial dilutions of serum were added to wells of a 96-well plate coated with purified recombinant human α-galactosidase A. Bound human α-galactosidase A-specific antibodies were detected with horseradish peroxidase-conjugated goat anti-rabbit immunoglobulin G, IgM, and IgA. A 30 minute incubation with calorimetric substrate was then used to detect rabbit anti-Ad2 antibodies. Anti-virus titers were defined as the reciprocal of the highest serum dilution that produced an OD490≧0.1.

As demonstrated in FIG. 10, AGAL expression was present in the serum of the rabbits throughout the 84 day time course. No detectable anti-AGAL antibodies were detected in the serum of the rabbits throughout the 84 day time course. The toxicities that resulted from delivering AAV2 were minor and generally similar in nature to those associated with Ad2-βgal delivery.

### EXAMPLE 7

### Catheter Based Delivery of an Adenovirus Gene Therapy Vector to the Rabbit Liver with an Extended Dwell Time

New Zealand white rabbits weighing approximately 4 kg each were used (Millbrook Farms, Amherst, Mass.). The adenoviral vector utilized, Ad2βgal, was described in Example 1. Each rabbit was injected with 1.5×10¹² viral particles/kg of the Ad2βgal virus.

Adenoviral gene therapy vector was delivered to the liver of rabbits utilizing the method described in Examples 1-3, with an 8 ml total injection volume, except that virus was allowed to dwell in the tissue for approximately four minutes rather than one minute.

Three days post-injection, the livers of treated rabbits were analyzed for beta-galactosidase expression. Bacterial beta-galactosidase expression in rabbit liver homogenates was quantified using a commercially available ELISA kit (Roche) per the manufacturer's instructions. Immunohistochemistry on tissue samples was performed as described in Example 1. Morphologic analysis as described in Example 1 was performed to determine the expression pattern in liver and the transfection ratio of liver hepatocytes to liver non-hepatocytes. For each rabbit, three sections of the injected lobe and three sections of the un-injected lobe were evaluated and the hepatocyte fraction [transfected hepatocytes/(transfected hepatocytes+transfected non-hepatocyte cells)] was determined.

As demonstrated in FIG. 11A, the extended dwell time significantly increased the proportion of transfected cells identified as hepatocytes in both the injected and un-injected lobes when compared to previous studies utilizing a 1 minute dwell time. Rabbits treated with adenoviral vector using the 4 minute dwell time had a hepatocyte fraction in the injected lobe of approximately 0.75-0.90 and a hepatocyte fraction in the un-injected lobe of approximately 0.70-0.90. (See bars in FIG. 11A representing 8-20 and 8-22.) In contrast, rabbits treated with adenoviral vector using an identical delivery method with a 1 minute dwell time had a transfected hepatocyte fraction of approximately 0.6-0.75 in the injected lobe and of approximately 0.30-0.70 in the un-injected lobe. (See bars in FIG. 11A representing 4, 5, and 1.) Interestingly, the increased dwell time did not appear to increase overall beta-galactosidase expression levels as shown in FIG. 11B.

### EXAMPLE 8

### Lobar Delivery with Outflow Blockade

The lobar delivery method, as described in Example 1, restricts delivery to a portion of the depot organ distal to the occlusion balloon. To further isolate the depot organ from systemic circulation, outflow blockade of all hepatic veins can be achieved by covering the hepatic venous ostia with a balloon catheter (6) deployed in the hepatic vena cava.

To perform this procedure, the femoral vein was accessed from the medial thigh via a longitudinal skin incision extending inferiorly from the femoral groove. Muscle fascia was bluntly dissected to expose the neurovascular bundle. The femoral vein was carefully dissected from the associated artery and nerve. A 1-2 cm segment of the femoral vein was isolated and ligated distally. A 7 French introducer sheath was inserted into the femoral vein, proximal to the ligation. A guidewire was advanced into the inferior vena cava using fluoroscopic guidance. A 5 French, 14 mm by 4 cm noncompliant balloon catheter was passed through the sheath over the guide wire into the hepatic portion of the vena cava. The outer diameter of the sheath is large relative to the rabbit femoral vein, making this procedure difficult to replicate in the rabbit model without vascular injury. This complication is not expected in human subjects.

The lumen of the catheter was heparinized. The balloon was inflated just prior to injection of the transfection agent via the balloon occlusion balloon catheter placed in a hepatic vein. Following inflation of the balloon, a small amount of radiographic contrast was injected through the introducer sheath to ensure that the balloon obstructed flow in the inferior vena cava. 8 ml of transfection agent was then injected through the endovascular catheter into a single isolated lobe. Immediately thereafter, the catheters and sheath were withdrawn and hemostasis was achieved. Dense radiographic contrast agent, as seen in FIG. 3, stained the isolated lobe, while more dilute contrast agent recirculated in a retrograde fashion to the remainder of the liver via the portal vein

### EXAMPLE 9

### Targeted Whole-Organ Delivery

To deliver a gene therapy agent to the entire liver with a single injection, the liver is isolated through the use of balloons inflated in the inferior vena cava both superior and inferior to the hepatic venous outflow. The transfection agent solution is then injected between the balloons and flows in a retrograde fashion through the hepatic veins to the entire hepatic parenchyma. One version of this method is shown in FIG. 4. In this version, balloon occlusion balloons (7, 8) are advanced from above through the jugular vein (5) to a position in the inferior vena cava (16) between the right atrium and the most superior hepatic vein (11), and from below through a femoral vein to a position in the inferior vena cava (16) between the most superior renal vein (19) and the most inferior hepatic vein (3). A 4 French pigtail catheter with multiple side holes near the tip is advanced through the opposite femoral vein to a position in the inferior vena cava between the two balloon occlusion balloons. The balloons are inflated to isolate the liver immediately prior to the injection of the gene therapy solution via the pigtail catheter.

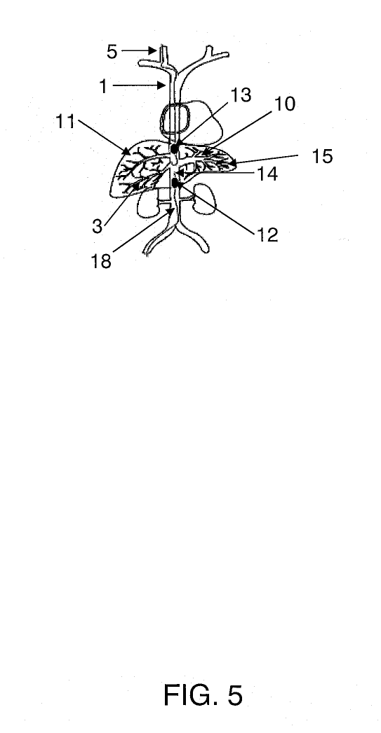

In an alternate embodiment of this method, the balloon occlusion balloons may be delivered via two separate dual-lumen balloon catheters (12, 13), as depicted in FIG. 5.

### EXAMPLE 10

### A Prophetic Study Using Local Catheter-Based Delivery of AAV8.DC190hAGA in Rhesus Monkeys with Varied Natural Immunity to AAV8

The efficacy of gene transfer and expression of human alpha-galactosidase following local, catheter-based delivery via the hepatic vein of an adeno-associated virus (AAV) serotype 8-based vector containing a prothrombin enhancer/human albumin promoter (DC190)-driven human alpha-galactosidase (hAGA) gene will be evaluated in Rhesus monkeys with a range of natural immunity to the AAV8 serotype.

Expression of hAGA resulting from local delivery will be compared to that obtained from systemic delivery via a peripheral vein. The level and duration of circulating alpha-galactosidase, generation of cytokines, and the presence of anti-AAV8 and anti-alpha-galactosidase antibodies will be examined. The tissue distribution of the transgene will also be determined.

Immunosuppression will likely be administered to animals prior to and following dosing with AAV8.DC190hAGA to minimize or eliminate the possibility that cytotoxic lymphocytes recognizing the viral capsid proteins might eliminate the transduced cells. Immunosuppressive agents that are commonly utilized in the field of organ transplantation may be used alone or in combination with other agents. Such immunosuppressive agents may include those used for induction and/or those used for maintenance. These agents may include cyclosporine (Neoral®), Sandimmune®), prednisone (Novo Prednisone®), Apo Prednisone®), azathioprine (Imuran®), tacrolimus or FK506 (Prograf®), mycophenolate mofetil (CellCept®), sirolimus (Rapamune®), OKT3 (Muromorab CO3®, Orthoclone®), ATGAM & Thymoglobulin. The immunosuppressive regime will likely comprise at least CELLCEPT® Oral Suspension and Rapamune® Oral Solution. CELLCEPT® Oral Suspension (MMF) will be given by nasal gavage at a dose of approximately 12.5 mg/kg twice daily. Rapamune® Oral Solution will be given by nasal gavage at a dose of approximately 2 mg/kg. These are thought to be the most reasonable estimates appropriate for rhesus monkeys

A single intravenous infusion of AAV8.DC190hAGA by a local-catheter-based approach and a systemic approach will be used. A dose level of approximately 2×10¹³ particles/kg of AAV8.DC190 hAGA is hypothesized to be sufficient to assess the pharmacokinetic and pharmacodynamic properties of the AAV8.DC190hAGA. The choice of dose level is based on information derived from previous studies conducted in rabbits and by other investigators with related materials in rhesus monkeys.

Monkeys will be screened for anti-AAV8 antibodies to determine the presence or absence of natural immunity to the AAV8 vector. AAV8 was originally isolated from monkeys, which is why the serotype is selected for use in the study. The use of a serotype isolated from the mammal sought to be treated may provide an advantage in increasing the transduction of the target organ. Such an increase may theoretically result from such a serotype because the serotype may have an increased tropism for the mammal's target organ based on the fact that the virus was isolated from the mammal. Use of monkeys with a range of natural immunity should also allow for the evaluation of the instant method in mammals with pre-existing immunity to the viral vector of choice.

Monkeys receiving virus via local catheter-based delivery will be treated using the method of the instant invention described in Examples 1 and 4. In brief, monkeys will receive virus via the catheter based procedure with the additional flushing step described in Example 4. The flushing step is theorized to dilute any anti-viral antibodies present in the target lobe, which should theoretically increase the efficiency of viral gene transfer and in particular, hepatocyte transduction. In addition, the dwell time of the virus will be increased from 1 minute as described in Examples 1 and 4 to a dwell time not to exceed 4 minutes. This increase in dwell time should theoretically increase the efficiency of viral gene transfer and in particular, hepatocyte transduction.

Study duration is estimated to be 365 days. Blood samples for evaluation of transgene expression, antibody levels, serum chemistry, and hematology parameters will be collected from all animals at various pre-determined time points throughout the course of the study.

The above study will evaluate the efficacy of gene transfer and expression of a therapeutic transgene delivered via a serotype originally isolated from monkeys. It will evaluate efficacy and expression in mammals with a natural range of pre-existing immunity to the serotype. The delivery method of the study will utilize a flushing step of the organ prior to virus administration and an extended dwell time for the virus. Immunosuppression will also be part of the study protocol. It is theorized that the efficacy and expression of the transgene using this methodology should produce equal, if not greater, target organ transduction than has been observed in the rabbit-based models. It is also theorized that the efficacy and expression of the transgene using this methodology should also produce equal, if not greater, target organ transduction than has been observed in other catheter-based systems in mammals that utilize anterograde delivery of viral vectors.

Since diseases can affect a variety of organs or tissues, it should be apparent that it would be desirable to use the methods of the present invention in various locations throughout the body. The present invention may be used to treat a variety of organs or tissues including the liver, kidney, heart, lungs, skeletal muscle, the stomach, or the intestines.

The specification is most thoroughly understood in light of the teachings of the references cited within the specification, all of which are hereby incorporated by reference in their entirety. The embodiments within the specification provide an illustration of embodiments of the invention and should not be construed to limit the scope of the invention. The skilled artisan recognizes that many other embodiments are encompassed by the claimed invention and that it is intended that the specification and examples be considered as exemplary only, with a true scope and spirit of the invention being indicated by the following claims.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method for delivering a viral gene therapy agent to a selected organ of a mammalian subject in order to express a protein encoded by the viral gene therapy agent, comprising:
a. placing one or more catheters within the venous vasculature which drains the organ; at least one of the catheters having one or more inflatably expandable members;
b. isolating an organ or section of an organ by occluding flow of fluids within the venous vasculature, which drains the organ or section of the organ, by inflating one or more of the inflatably expandable members;
c. delivering a viral gene therapy agent with a volume which causes a rise in vascular pressure of no more than 40% above the normal venous pressure in the isolated organ or isolated section of the organ;
d. allowing the gene therapy agent to persist within the isolated organ or isolated section of the organ for a period of time sufficient for transduction of a therapeutically effective amount of the agent.

2. The method of claim 1, wherein the mammalian subject is a human.

3. The method of claim 1, wherein said venous vasculature is a hepatic vein, a sublobar hepatic vein, or the inferior vena cava.

4. The method of claim 1, wherein the fraction of hepatocytes among hepatocytes plus non-hepatocytes, which are located in the isolated organ or isolated section of the organ and which express the protein encoded by the viral gene therapy agent is at least 0.2.

5. The method of claim 1, wherein the fraction of hepatocytes among hepatocytes plus non-hepatocytes, which are located in the isolated organ or isolated section of the organ and which express the protein encoded by the viral gene therapy agent is at least 0.3.

6. The method of claim 1, wherein the fraction of hepatocytes among hepatocytes plus non-hepatocytes, which are located in the isolated organ or isolated section of the organ and which express the protein encoded by the viral gene therapy agent is at least 0.4.

7. The method of claim 1, wherein the fraction of hepatocytes among hepatocytes plus non-hepatocytes which are located in the isolated organ or isolated section of the organ and which express the protein encoded by the viral gene therapy agent is at least 0.5.

8. The method of claim 1, wherein the fraction of hepatocytes among hepatocytes plus non-hepatocytes, which are located in the isolated organ or isolated section of the organ and which express the protein encoded by the viral gene therapy agent is at least 0.6.

9. The method of claim 1, wherein the organ is flushed with a solution prior to viral administration.

10. The method of claim 1 where the rise in vascular pressure is no more than 30% above the normal venous pressure in the isolated organ or isolated section of the organ.

11. The method of claim 1 where the rise in vascular pressure is no more than 20% above the normal venous pressure in the isolated organ or isolated section of the organ.

12. The method of claim 1 where the rise in vascular pressure is no more than 10% above the normal venous pressure in the isolated organ or isolated section of the organ.

13. The method of claim 1, wherein the catheter is a balloon occlusion catheter.

14. The method of claim 1, wherein the gene therapy agent is delivered via an endovascular catheter.

15. The method of claim 1, wherein the gene therapy agent is delivered via a percutaneous needle.

16. The method of claim 1, wherein the gene therapy agent comprises an adenoviral vector

17. The method of claim 1, wherein the gene therapy agent comprises an adeno-associated viral vector.

18. The method of claim 1, wherein the gene therapy agent comprises a lentiviral vector.

19. The method of claim 1, wherein the gene therapy agent comprises a retroviral vector.

20. The method of claim 1, wherein the gene therapy agent comprises a herpes viral vector.

21. The method of claim 1, wherein the gene therapy agent comprises an alpha viral vector.

22. The method of claim 1, wherein the gene therapy agent comprises a baculovirus vector.

23. The method of claim 1, wherein the gene therapy agent comprises a hybrid viral vector.

24. The method of claim 1, wherein the organ is the liver.

25. The method of claim 1, wherein the organ is a kidney.

26. The method of claim 24, wherein the liver is flushed with a solution prior to viral administration.

27. The method of claim 26, wherein the solution is saline.

28. The method of claim 7, wherein the organ is flushed with a solution prior to viral administration.

29. The method of claim 28, wherein the organ is the liver and the solution is saline.

30. The method of claim 1, wherein the dwell time is extended and increases the fraction of hepatocytes among hepatocytes plus non-hepatocytes, which are located in the isolated organ or isolated section of the organ and which express the protein encoded by the viral gene therapy agent to at least 0.8.

31. The method of claim 1, wherein the organ is flushed with a solution prior to viral administration and wherein step d) is extended and such extension increases the fraction of hepatocytes among hepatocytes plus non-hepatocytes, which are located in the isolated organ or isolated section of the organ and which express the protein encoded by the viral gene therapy agent to at least 0.8.

32. The method of claim 1, wherein step d) is from about 1 minute to about 4 minutes long.
